SEQUENCE LISTING (16 sequences)

SEQ ID NO: 1 - DNA, 1788 bp, Populus nigra
  CDS (1)..(1788)
atggcaactgaattattgtgcttgcaccgtccaatctcactgacacacaaattgttcagaaatcccttgcctaaagtcatccaggccactcccttaactttgaaactcagatgttctgtaagcacagaaaacgtcagcttcacagaaacagaaacagaaaccagaaggtctgccaattatgaaccaaatagctgggattatgattatttgctgtcttcggacactgacgaatcgattgaagtatacaaagacaaggccaaaaagctggaggctgaggtgagaagagagattaacaatgaaaaggcagagtttttgactctgcctgaactgatagataatgtccaaaggttaggattaggttaccggttcgagagtgacataaggagagcccttgatagatttgtttcttcaggaggatttgatgctgttacaaaaactagccttcatgctactgctcttagcttcaggcttctcagacagcatggctttgaggtctctcaagaagcgttcagcggattcaaggatcaaaatggcaatttcttgaaaaaccttaaggaggacatcaaggcaatactaagcctatatgaagcttcatttcttgccttagaaggagaaaatatcttggatgaggccaaggtgtttgcaatatcacatctaaaagagctcagcgaagaaaagattggaaaagacctggccgaacaggtgaatcatgcattggagcttccattgcatcgaaggacgcaaagactagaagctgtttggagcattgaagcataccgtaaaaaggaagatgcagatcaagtactgctagaacttgctatattggactacaacatgattcaatcagtataccaaagagatcttcgcgagacatcaaggtggtggaggcgtgtgggtcttgcaacaaagttgcattttgctagagacaggttaattgaaagcttttactgggcagttggagttgcgtttgaacctcaatacagtgattgccgtaattccgtagcaaaaatgttttcgtttgtaacaatcattgatgatatctatgatgtttatggtactctggatgagttggagctatttacagatgctgttgagagatgggatgttaatgccatcgatgatcttccggattatatgaagctctgcttcctagctctctataacactatcaatgagatagcttatgataatctgaaggacaagggggaaaacattcttccatacctaacaaaagcgtgggcagatttatgcaatgcattcctacaagaagcaaaatggttgtacaataagtccacaccaacatttgatgaatatttcggaaatgcatggaaatcatcctcagggcctcttcaactagtttttgcctactttgccgttgttcaaaacatcaagaaagaggaaattgataacttacaaaagtatcatgatatcatcagtaggccttcccacatctttcgtctttgcaacgacttggcttcagcatcggctgagatagcgagaggtgaaaccgcgaattctgtatcatgctacatgcgtacaaaaggcatttctgaggaacttgctactgaatccgtaatgaatttgatcgacgaaacctggaaaaagatgaacaaagaaaagcttggtggctctctgtttgcaaaaccttttgtcgaaacagctattaaccttgcacgacaatcccattgcacttatcacaacggagatgcgcatacttcaccagatgagctcactaggaaacgtgtcctgtcagtaatcacagagcctattctaccctttgagagataa

SEQ ID NO: 2 - protein, 595 aa, Populus nigra
MATELLCLHRPISLTHKLFRNPLPKVIQATPLTLKLRCSVSTENVSFTETETETRRSANYEPNSWDYDYLLSSDTDESIEVYKDKAKKLEAEVRREINNEKAEFLTLPELIDNVQRLGLGYRFESDIRRALDRFVSSGGFDAVTKTSLHATALSFRLLRQHGFEVSQEAFSGFKDQNGNFLKNLKEDIKAILSLYEASFLALEGENILDEAKVFAISHLKELSEEKIGKDLAEQVNHALELPLHRRTQRLEAVWSIEAYRKKEDADQVLLELAILDYNMIQSVYQRDLRETSRWWRRVGLATKLHFARDRLIESFYWAVGVAFEPQYSDCRNSVAKMFSFVTIIDDIYDVYGTLDELELFTDAVERWDVNAIDDLPDYMKLCFLALYNTINEIAYDNLKDKGENILPYLTKAWADLCNAFLQEAKWLYNKSTPTFDEYFGNAWKSSSGPLQLVFAYFAVVQNIKKEEIDNLQKYHDIISRPSHIFRLCNDLASASAEIARGETANSVSCYMRTKGISEELATESVMNLIDETWKKMNKEKLGGSLFAKPFVETAINLARQSHCTYHNGDAHTSPDELTRKRVLSVITEPILPFER

SEQ ID NO: 3 - DNA, 1746 bp, Lavandula angustifolia
  CDS (1)..(1746)
atgtctaccattattgcgatacaagtgttgcttcctattccaactactaaaacataccctagtcatgacttggagaagtcctcttcgcggtgtcgctcctcctccactcctcgccctagactgtgttgctcgttgcaggtgagtgatccgatcccaacgggccggcgatccggaggctacccgcccgccctatgggatttcgacactattcaatcgctcaacaccgagtataagggagagaggcacatgagaagggaagaagacctaattgggcaagttagagagatgctggtgcatgaagtagaggatcccactccacagctggagttcattgatgatttgcataagcttggcatatcttgccattttgagaatgaaatcctccaaatcttgaaatccatatatcttaatcaaaactacaaaagggatttgtactcaacatctctagcattcagactcctcagacaatatggcttcatccttccacaagaagtatttgattgtttcaagaatgaggagggtacggatttcaagccaagcttcggccgtgatatcaaaggcttgttacaattgtatgaagcttctttcctatcaagaaaaggagaagaaactttacaactagcaagagagtttgcaacaaagattctgcaaaaagaagttgatgagagagagtttgcaaccaagatggagttcccttctcattggacggttcaaatgccgaatgcaagacctttcatcgatgcttaccgtaggaggccggatatgaatccagttgtgctcgagctagccatacttgatacaaatatagttcaagcacaatttcaagaagaactcaaagagacctcaaggtggtgggagagtacaggcattgtccaagagcttccatttgtgagggataggattgtggaaggctacttttggacgattggagtgactcagagacgcgagcatggatacgaaagaatcatgaccgcaaaggttattgccttagtaacatgtttagacgacatatacgatgtttatggcacgatagaagagcttcaacttttcacaagcacaatccaaagatgggatttggaatcaatgaagcaactccctacctacatgcaagtaagctttcttgcactacacaactttgtaaccgaggtggcttacgatactctcaagaaaaagggctacaactccacaccatatttaagaaaaacgtgggtggatcttgttgaatcatatatcaaagaggcaacttggtactacaacggttataaacctagtatgcaagaataccttaacaatgcatggatatcagtcggaagtatggctatactcaaccacctcttcttccggttcacaaacgagagaatgcataaataccgcgatatgaaccgtgtctcgtccaacattgtgaggcttgctgatgatatgggaacatcattggctgaggtggagagaggggacgtgccgaaagcaattcaatgctacatgaatgagacgaatgcttctgaagaagaagcaagagaatatgtaagaagagtcatacaggaagaatgggaaaagttgaacacagaattgatgcgggatgatgatgatgatgatgattttacactatccaaatattactgtgaggtggttgctaatcttacaagaatggcacagtttatataccaagatggatcggatggcttcggcatgaaagattccaaggttaatagactgctaaaagagacgttgatcgagcgctacgaataa

SEQ ID NO: 4 - protein, 581 aa, Lavandula angustifolia
MSTIIAIQVLLPIPTTKTYPSHDLEKSSSRCRSSSTPRPRLCCSLQVSDPIPTGRRSGGYPPALWDFDTIQSLNTEYKGERHMRREEDLIGQVREMLVHEVEDPTPQLEFIDDLHKLGISCHFENEILQILKSIYLNQNYKRDLYSTSLAFRLLRQYGFILPQEVFDCFKNEEGTDFKPSFGRDIKGLLQLYEASFLSRKGEETLQLAREFATKILQKEVDEREFATKMEFPSHWTVQMPNARPFIDAYRRRPDMNPVVLELAILDTNIVQAQFQEELKETSRWWESTGIVQELPFVRDRIVEGYFWTIGVTQRREHGYERIMTAKVIALVTCLDDIYDVYGTIEELQLFTSTIQRWDLESMKQLPTYMQVSFLALHNFVTEVAYDTLKKKGYNSTPYLRKTWVDLVESYIKEATWYYNGYKPSMQEYLNNAWISVGSMAILNHLFFRFTNERMHKYRDMNRVSSNIVRLADDMGTSLAEVERGDVPKAIQCYMNETNASEEEAREYVRRVIQEEWEKLNTELMRDDDDDDDFTLSKYYCEVVANLTRMAQFIYQDGSDGFGMKDSKVNRLLKETLIERYE

SEQ ID NO: 5 - DNA, 1194 bp, Fusarium poae
  CDS (1)..(1194)
atggagaactttcccaccgagtattttctcaacacttctgtgcgccttcttgagtacattcgataccgagatagtaactacacccgagaagagcgcatcgagaatctgcactatgcttacaacaaggctgctcatcactttgctcagccccgacaacagcagctactcagggtagaccctaagcgactacaggcttccctccaaacaatcgttggcatggtcgtatacagttgggcaaaggtctctaaagagtgtatggcggctctatctattcattacacgtacacgctcgtcttggatgacagcagcgacgacccatatccagccatgatgaactatttcaacgatcttcaggccggacgagaacaggctcacccgtggtgggcgcttgtcaatgagcactttcccaatgtccttcgacattttggtcccttctgctcgttgaaccttatccgcagcactctcgactgtaagtacactggatacattgtttcaccacgacatgaaactgacagttatggaattaaagtttttgagggatgctggatcgagcagtacagctttggaggtttcccaggatcccatgactatcctcagtttcttcgacgcatgaatggcttgggtcactgtgtcggggcttctttgtggcccaaggagcagtttgatgaacgaagtctattccttgaaatcacatcagccattgctcagatggagaactggatggtctgggtcaacgatcttatgtcattctacaaggaattcgacgatgagcgtgaccagatcagcctcgtcaagaactacgtcgtctctgatgaaatcagtctccacgaagctttggagaagctgacccaagacactctgcactcgtccaagcagatggttgctgtcttctctgacaaggaccctcaggtgatggacacgattgagtgcttcatgcatggttatgtcacgtggcacttgtgcgatcacaggtaccgccttaatgagatctacgaaaaggtcaaggaacaaaagaccgaggacgctcagaagttttgcaagttttatgagcaggctgctaatgtcggtgccgtctcgccctcggagtgggcttacccaccagttgcgcaactggcaaacgttcgatccaaggatgtgaagaatgtgaagcagattgaaaagcctctgctgagttcaattgagctagtggagtga

SEQ ID NO: 6 - protein, 397 aa, Fusarium poae
MENFPTEYFLNTSVRLLEYIRYRDSNYTREERIENLHYAYNKAAHHFAQPRQQQLLRVDPKRLQASLQTIVGMVVYSWAKVSKECMAALSIHYTYTLVLDDSSDDPYPAMMNYFNDLQAGREQAHPWWALVNEHFPNVLRHFGPFCSLNLIRSTLDCKYTGYIVSPRHETDSYGIKVFEGCWIEQYSFGGFPGSHDYPQFLRRMNGLGHCVGASLWPKEQFDERSLFLEITSAIAQMENWMVWVNDLMSFYKEFDDERDQISLVKNYVVSDEISLHEALEKLTQDTLHSSKQMVAVFSDKDPQVMDTIECFMHGYVTWHLCDHRYRLNEIYEKVKEQKTEDAQKFCKFYEQAANVGAVSPSEWAYPPVAQLANVRSKDVKNVKQIEKPLLSSIELVE

SEQ ID NO: 7 - DNA, 480 bp, Thermococcus sp.
  CDS (1)..(480)
atgaaagttgaagctggtgattatgttctcttccactacgttggaaggttcgaggatggagaagtttttgacacaagctacgaggagatagccagagagaatggcattctcgtcgaggagagggagtacggcccaatgtgggtcaggataggcgtcggtgagatcatccctggcctcgatgaagccataattggcatggaagctggagagaagaagaccgtgaccgttccccccgagaaggcttacggaatgccgaacccagagcttgtaatctccgttccaagggaagaattcacaaaggccggccttgaaccccaggaaggtctctacgtcatgaccgattctggcatagccaagatagtttccgttggagagagcgaggtatcccttgacttcaaccacccgctagcaggtaagaccctagtctttgaggtagaagtcatagaagtaaaaaaggccgaagaggactcagaagcttag

SEQ ID NO: 8 - protein, 159 aa, Thermococcus sp.
MKVEAGDYVLFHYVGRFEDGEVFDTSYEEIARENGILVEEREYGPMWVRIGVGEIIPGLDEAIIGMEAGEKKTVTVPPEKAYGMPNPELVISVPREEFTKAGLEPQEGLYVMTDSGIAKIVSVGESEVSLDFNHPLAGKTLVFEVEVIEVKKAEEDSEA

SEQ ID NO: 9 - DNA, 3369 bp, Artificial
  Synthetic gene
catatgaaagttgaagctggtgattatgttctcttccactacgttggaaggttcgaggatggagaagtttttgacacaagctacgaggagatagccagagagaatggcattctcgtcgaggagagggagtacggcccaatgtgggtcaggataggcgtcggtgagatcatccctggcctcgatgaagccataattggcatggaagctggagagaagaagaccgtgaccgttccccccgagaaggcttacggaatgccgaacccagagcttgtaatctccgttccaagggaagaattcacaaaggccggccttgaaccccaggaaggtctctacgtcatgaccgattctggcatagccaagatagtttccgttggagagagcgaggtatcccttgacttcaaccacccgctagcaggtaagaccctagtctttgaggtagaagtcatagaagtaaaaaaggccgaagaggactcagaagctggaggtggaggttctggaggtggaggttctggaggtggaggttctggaggtggaggttctggaggtggatctgaaaccagaaggtctgccaattatgaaccaaatagctgggattatgattatttgctgtcttctgacactgacgaatctattgaagtatacaaagacaaggccaaaaagctggaggctgaggtgagaagagagattaacaatgaaaaggcagagtttttgactctgcctgaactgatagataatgttcaaaggttaggattaggttacagattcgagagtgacataaggagagcccttgatagatttgtttcttcaggaggatttgatgctgttacaaaaactagccttcatgctactgctcttagcttcaggcttctcagacagcatggctttgaggtatctcaagaagctttcagcggattcaaggatcaaaatggcaatttcttgaaaaaccttaaggaggacatcaaggcaatactaagcctatatgaagcttcatttcttgccttagaaggagaaaatatcttggatgaggccaaggtgtttgcaatatcacatctaaaagagcttagcgaagaaaagattggaaaagacctggccgaacaggtgaatcatgcattggagcttccattgcatagaaggacacaaagactagaagctgtttggagcattgaagcatacagaaaaaaggaagatgcagatcaagtactgctagaacttgctatattggactacaacatgattcaatcagtataccaaagagatcttagagagacatcaaggtggtggaggagagtgggtcttgcaacaaagttgcattttgctagagacaggttaattgaaagcttttactgggcagttggagttgcatttgaacctcaatacagtgattgtagaaattccgtagcaaaaatgttttcttttgtaacaatcattgatgatatctatgatgtttatggtactctggatgagttggagctatttacagatgctgttgagagatgggatgttaatgccatcgatgatcttcctgattatatgaagctttgtttcctagctctttataacactatcaatgagatagcttatgataatctgaaggacaagggggaaaacattcttccatacctaacaaaagcatgggcagatttatgtaatgcattcctacaagaagcaaaatggttgtacaataagtccacaccaacatttgatgaatatttcggaaatgcatggaaatcatcctcagggcctcttcaactagtttttgcctactttgccgttgttcaaaacatcaagaaagaggaaattgataacttacaaaagtatcatgatatcatcagtaggccttcccatatctttagactttgtaacgacttggcttcagcatctgctgagatagcaagaggtgaaaccgcaaattctgtatcatgttacatgagaacaaaaggcatttctgaggaacttgctactgaatccgtaatgaatttgatcgacgaaacctggaaaaagatgaacaaagaaaagcttggtggctctctgtttgcaaaaccttttgttgaaacagctattaaccttgcaagacaatcccattgtacttatcataacggagatgcacatacttcaccagatgagcttactaggaaaagagtactgtcagtaatcacagagcctattctaccttttgagagaggcggtagccaccatcaccatcaccattaataattttaaaatataagtgatttagatattcataatatatttgggaggtaaattaatatgaccagcgcccaacgtaaggacgaccatgtaaggctcgccatcgagcagcataacgcccatagcggacgtaaccagttcgacgacgtgtctttcgtacatcatgccctggccggcatcgacaggccagacgtgtccctggccacgtccttcgccgggatctcctggcaggtgcctatctacatcaacgcgatgaccggcggcagcgagaagaccggcctcatcaacagggacctggccaccgccgcccgtgagaccggcgtacccatcgcgtccgggtccatgaacgcgtacatcaaggacccctcctgcgccgacacgttccgtgtgctgcgtgacgagaaccccaacgggttcgtaatcgcgaacatcaacgccaccacgacggttgacaacgcgcagcgtgcgatcgacctgatcgaggcgaacgccctgcagatccatatcaacacggcgcaggagacgcctatgcctgagggcgacaggtctttcgcgtcctgggtccctcagatcgagaagatcgcggcggccgtagacatccccgtgatcgtaaaggaggtaggcaacggcctgagcaggcagaccatcctgctgctcgccgacctcggcgtgcaggcggcggacgtaagcggccgtggcggcacggacttcgcccgtatcgagaacggccgtagggagctcggcgactacgcgttcctgcatggctgggggcagtccaccgccgcctgcctgctggacgcccaggacatctccctgcccgtactcgcctccggcggtgtgcgtcatcctctcgacgtggtacgtgccctcgcgctcggcgcccgtgccgtaggctcctccgccggcttcctgcgtaccctgatggacgacggcgtagacgcgctgatcacgaagctcacgacctggctggaccagctggcggcgctgcagaccatgctcggcgcgcgtacccctgccgacctcacccgttgcgacgtgctgctccatggcgagctgcgtgacttctgcgccgacaggggcatcgacacgcgtcgtctcgcccagcgttccagctccatcgaggccctccagacgacgggaagcacaagataataaggatcc

SEQ ID NO: 10 - DNA, 2796 bp, Artificial
  Synthetic gene
catatggaaaccagaaggtctgccaattatgaaccaaatagctgggattatgattatttgctgtcttctgacactgacgaatctattgaagtatacaaagacaaggccaaaaagctggaggctgaggtgagaagagagattaacaatgaaaaggcagagtttttgactctgcctgaactgatagataatgttcaaaggttaggattaggttacagattcgagagtgacataaggagagcccttgatagatttgtttcttcaggaggatttgatgctgttacaaaaactagccttcatgctactgctcttagcttcaggcttctcagacagcatggctttgaggtatctcaagaagctttcagcggattcaaggatcaaaatggcaatttcttgaaaaaccttaaggaggacatcaaggcaatactaagcctatatgaagcttcatttcttgccttagaaggagaaaatatcttggatgaggccaaggtgtttgcaatatcacatctaaaagagcttagcgaagaaaagattggaaaagacctggccgaacaggtgaatcatgcattggagcttccattgcatagaaggacacaaagactagaagctgtttggagcattgaagcatacagaaaaaaggaagatgcagatcaagtactgctagaacttgctatattggactacaacatgattcaatcagtataccaaagagatcttagagagacatcaaggtggtggaggagagtgggtcttgcaacaaagttgcattttgctagagacaggttaattgaaagcttttactgggcagttggagttgcatttgaacctcaatacagtgattgtagaaattccgtagcaaaaatgttttcttttgtaacaatcattgatgatatctatgatgtttatggtactctggatgagttggagctatttacagatgctgttgagagatgggatgttaatgccatcgatgatcttcctgattatatgaagctttgtttcctagctctttataacactatcaatgagatagcttatgataatctgaaggacaagggggaaaacattcttccatacctaacaaaagcatgggcagatttatgtaatgcattcctacaagaagcaaaatggttgtacaataagtccacaccaacatttgatgaatatttcggaaatgcatggaaatcatcctcagggcctcttcaactagtttttgcctactttgccgttgttcaaaacatcaagaaagaggaaattgataacttacaaaagtatcatgatatcatcagtaggccttcccatatctttagactttgtaacgacttggcttcagcatctgctgagatagcaagaggtgaaaccgcaaattctgtatcatgttacatgagaacaaaaggcatttctgaggaacttgctactgaatccgtaatgaatttgatcgacgaaacctggaaaaagatgaacaaagaaaagcttggtggctctctgtttgcaaaaccttttgttgaaacagctattaaccttgcaagacaatcccattgtacttatcataacggagatgcacatacttcaccagatgagcttactaggaaaagagtactgtcagtaatcacagagcctattctaccttttgagagataataattttaaaatataagtgatttagatattcataatatatttgggaggtaaattaatatgaccagcgcccaacgtaaggacgaccatgtaaggctcgccatcgagcagcataacgcccatagcggacgtaaccagttcgacgacgtgtctttcgtacatcatgccctggccggcatcgacaggccagacgtgtccctggccacgtccttcgccgggatctcctggcaggtgcctatctacatcaacgcgatgaccggcggcagcgagaagaccggcctcatcaacagggacctggccaccgccgcccgtgagaccggcgtacccatcgcgtccgggtccatgaacgcgtacatcaaggacccctcctgcgccgacacgttccgtgtgctgcgtgacgagaaccccaacgggttcgtaatcgcgaacatcaacgccaccacgacggttgacaacgcgcagcgtgcgatcgacctgatcgaggcgaacgccctgcagatccatatcaacacggcgcaggagacgcctatgcctgagggcgacaggtctttcgcgtcctgggtccctcagatcgagaagatcgcggcggccgtagacatccccgtgatcgtaaaggaggtaggcaacggcctgagcaggcagaccatcctgctgctcgccgacctcggcgtgcaggcggcggacgtaagcggccgtggcggcacggacttcgcccgtatcgagaacggccgtagggagctcggcgactacgcgttcctgcatggctgggggcagtccaccgccgcctgcctgctggacgcccaggacatctccctgcccgtactcgcctccggcggtgtgcgtcatcctctcgacgtggtacgtgccctcgcgctcggcgcccgtgccgtaggctcctccgccggcttcctgcgtaccctgatggacgacggcgtagacgcgctgatcacgaagctcacgacctggctggaccagctggcggcgctgcagaccatgctcggcgcgcgtacccctgccgacctcacccgttgcgacgtgctgctccatggcgagctgcgtgacttctgcgccgacaggggcatcgacacgcgtcgtctcgcccagcgttccagctccatcgaggccctccagacgacgggaagcacaagataataaggatcc

SEQ ID NO: 11 - DNA, 3726 bp, Artificial
  Synthetic gene
cccgggttcagattaaatttttgcttatttgatttacattatataatattgagtaaagtattgactagcaaaattttttgatactttaatttgtgaaatttcttatcaaaagttatatttttgaatgatttttattgaaaaatacaactaaaaaggattatagtataagtgtgtgtaattttgtgttaaatttaaagggaggaaatgaacatgaaagttgaagctggtgattatgttctcttccactacgttggaaggttcgaggatggagaagtttttgacacaagctacgaggagatagccagagagaatggcattctcgtcgaggagagggagtacggcccaatgtgggtcaggataggcgtcggtgagatcatccctggcctcgatgaagccataattggcatggaagctggagagaagaagaccgtgaccgttccccccgagaaggcttacggaatgccgaacccagagcttgtaatctccgttccaagggaagaattcacaaaggccggccttgaaccccaggaaggtctctacgtcatgaccgattctggcatagccaagatagtttccgttggagagagcgaggtatcccttgacttcaaccacccgctagcaggtaagaccctagtctttgaggtagaagtcatagaagtaaaaaaggccgaagaggactcagaagctggaggtggaggttctggaggtggaggttctggaggtggaggttctggaggtggaggttctggaggtggatctgaaaccagaaggtctgccaattatgaaccaaatagctgggattatgattatttgctgtcttctgacactgacgaatctattgaagtatacaaagacaaggccaaaaagctggaggctgaggtgagaagagagattaacaatgaaaaggcagagtttttgactctgcctgaactgatagataatgttcaaaggttaggattaggttacagattcgagagtgacataaggagagcccttgatagatttgtttcttcaggaggatttgatgctgttacaaaaactagccttcatgctactgctcttagcttcaggcttctcagacagcatggctttgaggtatctcaagaagctttcagcggattcaaggatcaaaatggcaatttcttgaaaaaccttaaggaggacatcaaggcaatactaagcctatatgaagcttcatttcttgccttagaaggagaaaatatcttggatgaggccaaggtgtttgcaatatcacatctaaaagagcttagcgaagaaaagattggaaaagacctggccgaacaggtgaatcatgcattggagcttccattgcatagaaggacacaaagactagaagctgtttggagcattgaagcatacagaaaaaaggaagatgcagatcaagtactgctagaacttgctatattggactacaacatgattcaatcagtataccaaagagatcttagagagacatcaaggtggtggaggagagtgggtcttgcaacaaagttgcattttgctagagacaggttaattgaaagcttttactgggcagttggagttgcatttgaacctcaatacagtgattgtagaaattccgtagcaaaaatgttttcttttgtaacaatcattgatgatatctatgatgtttatggtactctggatgagttggagctatttacagatgctgttgagagatgggatgttaatgccatcgatgatcttcctgattatatgaagctttgtttcctagctctttataacactatcaatgagatagcttatgataatctgaaggacaagggggaaaacattcttccatacctaacaaaagcatgggcagatttatgtaatgcattcctacaagaagcaaaatggttgtacaataagtccacaccaacatttgatgaatatttcggaaatgcatggaaatcatcctcagggcctcttcaactagtttttgcctactttgccgttgttcaaaacatcaagaaagaggaaattgataacttacaaaagtatcatgatatcatcagtaggccttcccatatctttagactttgtaacgacttggcttcagcatctgctgagatagcaagaggtgaaaccgcaaattctgtatcatgttacatgagaacaaaaggcatttctgaggaacttgctactgaatccgtaatgaatttgatcgacgaaacctggaaaaagatgaacaaagaaaagcttggtggctctctgtttgcaaaaccttttgttgaaacagctattaaccttgcaagacaatcccattgtacttatcataacggagatgcacatacttcaccagatgagcttactaggaaaagagtactgtcagtaatcacagagcctattctaccttttgagagaggcggtagccaccatcaccatcaccattaataattttaaaatataagtgatttagatattcataatatatttgggaggtaaattaatatgaccagcgcccaacgtaaggacgaccatgtaaggctcgccatcgagcagcataacgcccatagcggacgtaaccagttcgacgacgtgtctttcgtacatcatgccctggccggcatcgacaggccagacgtgtccctggccacgtccttcgccgggatctcctggcaggtgcctatctacatcaacgcgatgaccggcggcagcgagaagaccggcctcatcaacagggacctggccaccgccgcccgtgagaccggcgtacccatcgcgtccgggtccatgaacgcgtacatcaaggacccctcctgcgccgacacgttccgtgtgctgcgtgacgagaaccccaacgggttcgtaatcgcgaacatcaacgccaccacgacggttgacaacgcgcagcgtgcgatcgacctgatcgaggcgaacgccctgcagatccatatcaacacggcgcaggagacgcctatgcctgagggcgacaggtctttcgcgtcctgggtccctcagatcgagaagatcgcggcggccgtagacatccccgtgatcgtaaaggaggtaggcaacggcctgagcaggcagaccatcctgctgctcgccgacctcggcgtgcaggcggcggacgtaagcggccgtggcggcacggacttcgcccgtatcgagaacggccgtagggagctcggcgactacgcgttcctgcatggctgggggcagtccaccgccgcctgcctgctggacgcccaggacatctccctgcccgtactcgcctccggcggtgtgcgtcatcctctcgacgtggtacgtgccctcgcgctcggcgcccgtgccgtaggctcctccgccggcttcctgcgtaccctgatggacgacggcgtagacgcgctgatcacgaagctcacgacctggctggaccagctggcggcgctgcagaccatgctcggcgcgcgtacccctgccgacctcacccgttgcgacgtgctgctccatggcgagctgcgtgacttctgcgccgacaggggcatcgacacgcgtcgtctcgcccagcgttccagctccatcgaggccctccagacgacgggaagcacaagataataatttaaaagcaaatataaatgaaaaattgaaccctagcattatgtaaatgcagggtttaatttttatattaagcagcataatagaaagttttttaaatgcatgtatatatggggtatttaaagggaaatctataatataatttaggactattccgga

SEQ ID NO: 12 - DNA, 3153 bp, Artificial
  Synthetic gene
cccgggttcagattaaatttttgcttatttgatttacattatataatattgagtaaagtattgactagcaaaattttttgatactttaatttgtgaaatttcttatcaaaagttatatttttgaatgatttttattgaaaaatacaactaaaaaggattatagtataagtgtgtgtaattttgtgttaaatttaaagggaggaaatgaacatggaaaccagaaggtctgccaattatgaaccaaatagctgggattatgattatttgctgtcttctgacactgacgaatctattgaagtatacaaagacaaggccaaaaagctggaggctgaggtgagaagagagattaacaatgaaaaggcagagtttttgactctgcctgaactgatagataatgttcaaaggttaggattaggttacagattcgagagtgacataaggagagcccttgatagatttgtttcttcaggaggatttgatgctgttacaaaaactagccttcatgctactgctcttagcttcaggcttctcagacagcatggctttgaggtatctcaagaagctttcagcggattcaaggatcaaaatggcaatttcttgaaaaaccttaaggaggacatcaaggcaatactaagcctatatgaagcttcatttcttgccttagaaggagaaaatatcttggatgaggccaaggtgtttgcaatatcacatctaaaagagcttagcgaagaaaagattggaaaagacctggccgaacaggtgaatcatgcattggagcttccattgcatagaaggacacaaagactagaagctgtttggagcattgaagcatacagaaaaaaggaagatgcagatcaagtactgctagaacttgctatattggactacaacatgattcaatcagtataccaaagagatcttagagagacatcaaggtggtggaggagagtgggtcttgcaacaaagttgcattttgctagagacaggttaattgaaagcttttactgggcagttggagttgcatttgaacctcaatacagtgattgtagaaattccgtagcaaaaatgttttcttttgtaacaatcattgatgatatctatgatgtttatggtactctggatgagttggagctatttacagatgctgttgagagatgggatgttaatgccatcgatgatcttcctgattatatgaagctttgtttcctagctctttataacactatcaatgagatagcttatgataatctgaaggacaagggggaaaacattcttccatacctaacaaaagcatgggcagatttatgtaatgcattcctacaagaagcaaaatggttgtacaataagtccacaccaacatttgatgaatatttcggaaatgcatggaaatcatcctcagggcctcttcaactagtttttgcctactttgccgttgttcaaaacatcaagaaagaggaaattgataacttacaaaagtatcatgatatcatcagtaggccttcccatatctttagactttgtaacgacttggcttcagcatctgctgagatagcaagaggtgaaaccgcaaattctgtatcatgttacatgagaacaaaaggcatttctgaggaacttgctactgaatccgtaatgaatttgatcgacgaaacctggaaaaagatgaacaaagaaaagcttggtggctctctgtttgcaaaaccttttgttgaaacagctattaaccttgcaagacaatcccattgtacttatcataacggagatgcacatacttcaccagatgagcttactaggaaaagagtactgtcagtaatcacagagcctattctaccttttgagagataataattttaaaatataagtgatttagatattcataatatatttgggaggtaaattaatatgaccagcgcccaacgtaaggacgaccatgtaaggctcgccatcgagcagcataacgcccatagcggacgtaaccagttcgacgacgtgtctttcgtacatcatgccctggccggcatcgacaggccagacgtgtccctggccacgtccttcgccgggatctcctggcaggtgcctatctacatcaacgcgatgaccggcggcagcgagaagaccggcctcatcaacagggacctggccaccgccgcccgtgagaccggcgtacccatcgcgtccgggtccatgaacgcgtacatcaaggacccctcctgcgccgacacgttccgtgtgctgcgtgacgagaaccccaacgggttcgtaatcgcgaacatcaacgccaccacgacggttgacaacgcgcagcgtgcgatcgacctgatcgaggcgaacgccctgcagatccatatcaacacggcgcaggagacgcctatgcctgagggcgacaggtctttcgcgtcctgggtccctcagatcgagaagatcgcggcggccgtagacatccccgtgatcgtaaaggaggtaggcaacggcctgagcaggcagaccatcctgctgctcgccgacctcggcgtgcaggcggcggacgtaagcggccgtggcggcacggacttcgcccgtatcgagaacggccgtagggagctcggcgactacgcgttcctgcatggctgggggcagtccaccgccgcctgcctgctggacgcccaggacatctccctgcccgtactcgcctccggcggtgtgcgtcatcctctcgacgtggtacgtgccctcgcgctcggcgcccgtgccgtaggctcctccgccggcttcctgcgtaccctgatggacgacggcgtagacgcgctgatcacgaagctcacgacctggctggaccagctggcggcgctgcagaccatgctcggcgcgcgtacccctgccgacctcacccgttgcgacgtgctgctccatggcgagctgcgtgacttctgcgccgacaggggcatcgacacgcgtcgtctcgcccagcgttccagctccatcgaggccctccagacgacgggaagcacaagataataatttaaaagcaaatataaatgaaaaattgaaccctagcattatgtaaatgcagggtttaatttttatattaagcagcataatagaaagttttttaaatgcatgtatatatggggtatttaaagggaaatctataatataatttaggactattccgga

SEQ ID NO: 13 - DNA, 3897 bp, Artificial
  Plasmid vector pSKCL
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagaattcggcgcgcccgggccgcggatccatatggtacctctagacgcgtgcatgctcgagtccggaagcttttcagattaaatttttgcttatttgatttacattatataatattgagtaaagtattgactagcaaaattttttgatactttaatttgtgaaatttcttatcaaaagttatatttttgaatgatttttattgaaaaatacaactaaaaaggattatagtataagtgtgtgtaattttgtgttaaatttaaagggaggaaatgaacatgaactttaataaaattgatttagacaattggaagagaaaagagatatttaatcattatttgaaccaacaaacgacttttagtataaccacagaaattgatattagtgttttatacagaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatttaaaagcaaatataaatgaaaaattgaaccctagcattatgtaaatgcagggtttaatttttatattaagcagcataatagaaagttttttaaatgcatgtatatatggggtatttaaagggaaatctataatataatttaggactattcgaattttttattaaaacgtctcaaaatcgtttctgagacgttttagcgtttatttcgtttagttatcggcataatcgttaaaacaggcgttatcgtagcgtaaaagcccttgagcgtagcgtggctttgcagcgaagatgttgtctgttagattatgaaagccgatgactgaatgaaataataagcgcagcgcccttctatttcggttggaggaggctcaagggagtatgagggaatgaaattccctcatgggtttgattttaaaaattgcttgcaattttgccgagcggtagcgctggaaaatttttgaaaaaaatttggaatttggaaaaaatttggtttgatgttgcgattaatagcaatacaattgcaataaacaaaatgatcgatgctgtttggcaaaaaaagaaaaagtgattaatttatattttatttatggcgctaatttattacggctttttttgttgtcggctagccgattctgatacatttttttaagcacaaaaaccacccaattttggagtggtgtgtaagtgcgcattgtcatgaaaaaatggcacgcaatttcatcactttttaaagtgatgtgtaagtgcgcattgtcatgaaaaaatggcacgcaatttcatcactttttaaagtgatgtgtaagtgcgcattgttggaaaaatcgaactatgatttatttttgctgttgtatttatttttcatcttttgggttttggttttgttttttgttgctatcgtagtttatttgctttttaagggctctatttttcgttctacggcatttttataatttgccaatataatttat

SEQ ID NO: 14 - DNA, 2509 bp, Artificial
  Synthetic gene
ggatccttcagattaaatttttgcttatttgatttacattatataatattgagtaaagtattgactagcaaaattttttgatactttaatttgtgaaatttcttatcaaaagttatatttttgaatgatttttattgaaaaatacaactaaaaaggattatagtataagtgtgtgtaattttgtgttaaatttaaagggaggaaatgaacatgaaagttgaagctggtgattatgttctcttccactacgttggaaggttcgaggatggagaagtttttgacacaagctacgaggagatagccagagagaatggcattctcgtcgaggagagggagtacggcccaatgtgggtcaggataggcgtcggtgagatcatccctggcctcgatgaagccataattggcatggaagctggagagaagaagaccgtgaccgttccccccgagaaggcttacggaatgccgaacccagagcttgtaatctccgttccaagggaagaattcacaaaggccggccttgaaccccaggaaggtctctacgtcatgaccgattctggcatagccaagatagtttccgttggagagagcgaggtatcccttgacttcaaccacccgctagcaggtaagaccctagtctttgaggtagaagtcatagaagtaaaaaaggccgaagaggactcagaagctggaggtggaggttctggaggtggaggttctggaggtggaggttctggaggtggaggttctggaggtggatctacgggccggcgatccggaggctacccgcccgccctatgggatttcgacactattcaatcgctcaacaccgagtataagggagagaggcacatgagaagggaagaagacctaattgggcaagttagagagatgctggtgcatgaagtagaagatcccactccacagctggagttcattgatgatttgcataagcttggcatatcttgccattttgagaatgaaatcctccaaatcttgaaatccatatatcttaatcaaaactacaaaagggatttgtactcaacatctctagcattcagactcctcagacaatatggcttcatccttccacaagaagtatttgattgtttcaagaatgaggagggtacggatttcaagccaagcttcggccgtgatatcaaaggcttgttacaattgtatgaagcttctttcctatcaagaaaaggagaagaaactttacaactagcaagagagtttgcaacaaagattctgcaaaaagaagttgatgagagagagtttgcaaccaagatggagttcccttctcattggacggttcaaatgccgaatgcaagacctttcatcgatgcttaccgtaggaggccggatatgaatccagttgtgctcgagctagccatacttgatacaaatatagttcaagcacaatttcaagaagaactcaaagagacctcaaggtggtgggagagtacaggcattgtccaagagcttccatttgtgagggataggattgtggaaggctacttttggacgattggagtgactcagagacgcgagcatggatacgaaagaatcatgaccgcaaaggttattgccttagtaacatgtttagacgacatatacgatgtttatggcacgatagaagagcttcaacttttcacaagcacaatccaaagatgggatttggaatcaatgaagcaactccctacctacatgcaagtaagctttcttgcactacacaactttgtaaccgaggtggcttacgatactctcaagaaaaagggctacaactccacaccatatttaagaaaaacgtgggtggatcttgttgaatcatatatcaaagaggcaacttggtactacaacggttataaacctagtatgcaagaataccttaacaatgcatggatatcagtcggaagtatggctatactcaaccacctcttcttccggttcacaaacgagagaatgcataaataccgcgatatgaaccgtgtctcgtccaacattgtgaggcttgctgatgatatgggaacatcattggctgaggtggagagaggggacgtgccgaaagcaattcaatgctacatgaatgagacgaatgcttctgaagaagaagcaagagaatatgtaagaagagtcatacaggaagaatgggaaaagttgaacacagaattgatgcgggatgatgatgatgatgatgattttacactatccaaatattactgtgaggtggttgctaatcttacaagaatggcacagtttatataccaagatggatcggatggcttcggcatgaaagattccaaggttaatagactgctaaaagagacgttgatcgagcgctacgaataataatttaaaagcaaatataaatgaaaaattgaaccctagcattatgtaaatgcagggtttaatttttatattaagcagcataatagaaagttttttaaatgcatgtatatatggggtatttaaagggaaatctataatataatttaggactattggtacc

SEQ ID NO: 15 - DNA, 7244 bp, Artificial
  Plasmid vector pCM80
gaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctagcttcccgcttggtcgggccgcttcgcgagggcccgttgacgacaacggtgcgatgggtcccggccccggtcaagacgatgccaatacgttgcgacactacgccttggcacttttagaattgccttatcgtcctgataagaaatgtccgaccagctaaagacatcgcgtccaatcaaagcctagaaaatataggcgaagggacgctaataagtctttcataagaccgcgcaaatctaaaaatatccttagattcacgatgcggcacttcggatgacttccgagcgagcctggaacctcagaaaaacgtctgagagataccgcggatctcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgattgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgt

SEQ ID NO: 16 - DNA, 1963 bp, Artificial
  Synthetic gene
ggatccttcagattaaatttttgcttatttgatttacattatataatattgagtaaagtattgactagcaaaattttttgatactttaatttgtgaaatttcttatcaaaagttatatttttgaatgatttttattgaaaaatacaactaaaaaggattatagtataagtgtgtgtaattttgtgttaaatttaaagggaggaaatgaacatgacgggccggcgatccggaggctacccgcccgccctatgggatttcgacactattcaatcgctcaacaccgagtataagggagagaggcacatgagaagggaagaagacctaattgggcaagttagagagatgctggtgcatgaagtagaagatcccactccacagctggagttcattgatgatttgcataagcttggcatatcttgccattttgagaatgaaatcctccaaatcttgaaatccatatatcttaatcaaaactacaaaagggatttgtactcaacatctctagcattcagactcctcagacaatatggcttcatccttccacaagaagtatttgattgtttcaagaatgaggagggtacggatttcaagccaagcttcggccgtgatatcaaaggcttgttacaattgtatgaagcttctttcctatcaagaaaaggagaagaaactttacaactagcaagagagtttgcaacaaagattctgcaaaaagaagttgatgagagagagtttgcaaccaagatggagttcccttctcattggacggttcaaatgccgaatgcaagacctttcatcgatgcttaccgtaggaggccggatatgaatccagttgtgctcgagctagccatacttgatacaaatatagttcaagcacaatttcaagaagaactcaaagagacctcaaggtggtgggagagtacaggcattgtccaagagcttccatttgtgagggataggattgtggaaggctacttttggacgattggagtgactcagagacgcgagcatggatacgaaagaatcatgaccgcaaaggttattgccttagtaacatgtttagacgacatatacgatgtttatggcacgatagaagagcttcaacttttcacaagcacaatccaaagatgggatttggaatcaatgaagcaactccctacctacatgcaagtaagctttcttgcactacacaactttgtaaccgaggtggcttacgatactctcaagaaaaagggctacaactccacaccatatttaagaaaaacgtgggtggatcttgttgaatcatatatcaaagaggcaacttggtactacaacggttataaacctagtatgcaagaataccttaacaatgcatggatatcagtcggaagtatggctatactcaaccacctcttcttccggttcacaaacgagagaatgcataaataccgcgatatgaaccgtgtctcgtccaacattgtgaggcttgctgatgatatgggaacatcattggctgaggtggagagaggggacgtgccgaaagcaattcaatgctacatgaatgagacgaatgcttctgaagaagaagcaagagaatatgtaagaagagtcatacaggaagaatgggaaaagttgaacacagaattgatgcgggatgatgatgatgatgatgattttacactatccaaatattactgtgaggtggttgctaatcttacaagaatggcacagtttatataccaagatggatcggatggcttcggcatgaaagattccaaggttaatagactgctaaaagagacgttgatcgagcgctacgaataataatttaaaagcaaatataaatgaaaaattgaaccctagcattatgtaaatgcagggtttaatttttatattaagcagcataatagaaagttttttaaatgcatgtatatatggggtatttaaagggaaatctataatataatttaggactattggtacc

(19) United States
(12) Patent Application Publication
(10) Pub. No.: US 2018/0023098 A1
(43) Pub. Date: Jan. 25, 2018

(54) NUCLEIC ACID, FUSION PROTEIN, RECOMBINED CELL, AND ISOPRENE OR CYCLIC TERPENE PRODUCTION METHOD

(71) Applicant: SEKISUI CHEMICAL CO., LTD., Osaka (JP)

(72) Inventors: Masahiro FURUTANI, Arakawa-ku, Tokyo (JP); Kazufumi KAWABATA, Tsukuba-shi, Ibaraki (JP); Norihide NISHIYAMA, Tsurugashima-shi, Saitama (JP)

(73) Assignee: SEKISUI CHEMICAL CO., LTD., Osaka (JP)

(21) Appl. No.: 15/549,739

(22) PCT Filed: Feb. 10, 2016
(86) PCT No.: PCT/JP2016/053977

§ 371 Date: Aug. 9, 2017

(30) Foreign Application Priority Data
Feb. 13, 2015 (JP) 2015-026156
Nov. 6, 2015 (JP) 2015-218114

(51) Int. Cl. C12P 5/02; C12P 5/00; C12N 9/88; C12N 15/62; C12N 9/90

(52) U.S. Cl. CPC C12P 5/026; C12N 15/62; C12N 9/90; C12Y 502/01008; C12N 9/88; C12Y 402/03027; C12Y 402/03052; C12P 5/002

(57) ABSTRACT

Provided is a nucleic acid encoding a fusion protein, the fusion protein including a first protein selected from the group consisting of isoprene synthase and cyclic terpene synthase, and a FKBP family protein linked to the first protein. Provided is a fusion protein encoded by the nucleic acid. Provided is a recombinant cell including the nucleic acid and expressing the fusion protein. Further provided is a recombinant cell including a first nucleic acid encoding the first protein and a second nucleic acid encoding the FKBP family protein, and expressing the first protein and the FKBP family protein. As a host cell, a syngas-assimilating bacterium or a methanol assimilating bacterium can be used.

### TECHNICAL FIELD

The present invention relates to a nucleic acid, a fusion protein, a recombinant cell, and a method for producing isoprene or cyclic terpene. In particular, the present invention relates to a nucleic acid encoding a fusion protein that includes isoprene synthase or cyclic terpene synthase and a FKBP family protein, the fusion protein, a recombinant cell including the nucleic acid and the like, and a method for producing isoprene or cyclic terpene using the recombinant cell.

### BACKGROUND ART

Isoprene is a monomer raw material for synthetic polyisoprene, and is an important material, in particular, in the tire industry. In recent years, development and commercialization has been steadily progressing in the technique for conversion from a production process of basic chemicals depending on petroleum to a production process from renewable resources such as plant resources.

Isoprene synthase (EC 4.2.3.27) has action of converting dimethylallyl diphosphate (DMAPP) as an isomer of isopentenyl diphosphate (IPP) into isoprene.

A method for producing isoprene using a recombinant cell (a recombinant) is known. For example, in the inventions described in Patent Documents 1 and 2, isoprene is produced using a recombinant cell into which a nucleic acid (gene) encoding isoprene synthase has been introduced. In detail, the recombinant cell is cultured using methanol or syngas (synthesis gas) as a carbon source, and isoprene is obtained from the cultured product. Furthermore, a production technique by recombinant Escherichia coli using sugar as a raw material is known (see, for example, Patent Documents 3 and 4).

Terpene is a generic name of compounds having 10 or more carbon atoms in which two or more molecules of isopentenyl diphosphate (IPP) as an isoprene unit (C5) are linked to each other by the action of prenyltransferase. Terpenes are classified into monoterpene (C10), sesquiterpene (C15), diterpene (C20), triterpene (C30), tetraterpene (C40), and the like, depending on the number of isoprene units. Inter alia, many cyclic monoterpenes, cyclic sesquiterpenes, and cyclic diterpenes are useful as perfume raw materials, cosmetic raw materials, pharmaceutical intermediates, adhesive raw materials, and high function resin raw materials.

An enzyme for producing cyclic terpene using linear terpene as a substrate is referred to as cyclic terpene synthase. Cyclic terpene is synthesized by cyclization of linear terpene by the action of the cyclic terpene synthase. For example, cyclic monoterpene is synthesized as follows, that is, for example, geranyl diphosphate (GPP) generates by biomolecular binding of IPP, and is formed into a cyclic structure by action of cyclic monoterpene synthase. The cyclic sesquiterpene is synthesized by cyclization of famesyl diphosphate (C15: FPP) as a precursor by the action of cyclic sesquiterpene synthase. The cyclic diterpene is synthesized by cyclization of geranylgeranyl diphosphate (C20: GGPP) as a precursor by the action of cyclic diterpene synthase.

Well-known examples of the method for producing cyclic terpene using a recombinant cell (a recombinant) include a method for producing β-phellandrene as one type of cyclic monoterpene (Patent Document 5). In this method, β-phellandrene is produced by converting geranyl diphosphate (GPP) or neryl diphosphate (NPP) into β-phellandrene by using a recombinant cell into which a nucleic acid encoding β-phellandrene synthase has been introduced. The β-phellandrene has a β-phellandrene conjugated diene structure, and therefore is useful natural monomer for polymerization.

### PRIOR ART DOCUMENTS

### Patent Documents

Patent Document 1: WO2014/065271

Patent Document 2: WO02014/104202

Patent Document 3: WO2009/076676

Patent Document 4: WO2009/132220

Patent Document 5: JP 2014-76042 A

### DISCLOSURE OF INVENTION

### Technical Problem

Most of the currently available isoprene synthase and cyclic terpene synthase are derived from plants. Therefore, when they are expressed, in particular, in a bacterial host cell, three-dimensional structure cannot be formed successfully in the cytoplasm, causing problems such as degradation by host protease, or insolubilization (Patent Documents 3 and 4). In order to improve productivity of isoprene or cyclic terpene, improvement of stability of isoprene synthase and cyclic terpene synthase in a recombinant cell has been demanded.

An object of the present invention is to provide a technique for improving the stability of isoprene synthase and cyclic terpene synthase in a recombinant cell, and improving the productivity of isoprene and cyclic terpene by a recombinant cell.

### Solution to Problem

The inventors have succeeded in improving the stability of isoprene synthase and cyclic terpene synthase in a recombinant cell by using a FKBP family protein as one type of molecular chaperone. Then, the inventors have succeeded in improving the productivity of isoprene and cyclic terpene by using the recombinant cell, reaching the completion of the present invention.

One aspect of the present invention relates to a nucleic acid encoding a fusion protein, the fusion protein including: a first protein selected from the group consisting of isoprene synthase and cyclic terpene synthase; and a FKBP family protein linked to the first protein.

The nucleic acid of this aspect encodes a fusion protein (chimeric protein) which includes a first protein selected from the group consisting of isoprene synthase and cyclic terpene synthase, and a FKBP family protein linked to the first protein. Since in the fusion protein, isoprene synthase or cyclic terpene synthase is stabilized by the action of FKBP family protein adjacent thereto in the same molecule, the isoprene synthase activity or the cyclic terpene synthase activity is exhibited more stably. Therefore, for example, when the fusion protein is expressed in a recombinant cell into which the nucleic acid of this aspect has been introduced, degradation by host protease, or insolubilization is less likely to occur in the recombinant cell as compared with the case where isoprene synthase or cyclic terpene synthase is singly used.

Preferably, the first protein is isoprene synthase.

The nucleic acid of this aspect encodes a fusion protein which includes isoprene synthase and a FKBP family protein linked thereto. Since in the fusion protein, isoprene synthase is stabilized by the action of FKBP family protein adjacent thereto in the same molecule, the isoprene synthase activity is exhibited more stably. Therefore, for example, when the fusion protein is expressed in a recombinant cell into which the nucleic acid of this aspect has been introduced, degradation by host protease, or insolubilization is less likely to occur in the recombinant cell as compared with the case where isoprene synthase is singly used.

Preferably, the isoprene synthase is any one of the following (a-1) to (a-3):

(a-1) a protein consisting of the amino acid sequence of SEQ ID NO: 2,
(a-2) a protein consisting of the amino acid sequence in which 1 to 20 amino acids are deleted, substituted or added in the amino acid sequence of SEQ ID NO: 2, and having isoprene synthase activity, and
(a-3) a protein consisting of an amino acid sequence having homology of 90% or more with the amino acid sequence of SEQ ID NO: 2, and having isoprene synthase activity.

Preferably, the first protein is cyclic monoterpene synthase.

Preferably, the cyclic monoterpene synthase is phellandrene synthase.

Preferably, the cyclic monoterpene synthase is any one of the following (b-1) to (b-3):

(b-1) a protein consisting of the amino acid sequence of SEQ ID NO: 4,
(b-2) a protein consisting of the amino acid sequence in which 1 to 20 amino acids are deleted, substituted or added in the amino acid sequence of SEQ ID NO: 4, and having β-phellandrene synthase activity, and
(b-3) a protein consisting of an amino acid sequence having homology of 90% or more with the amino acid sequence of SEQ ID NO: 4, and having i-phellandrene synthase activity.

Preferably, the first protein is cyclic sesquiterpene synthase.

Preferably, the cyclic sesquiterpene synthase is any one of the following (c-1) to (c-3):

(c-1) a protein consisting of the amino acid sequence of SEQ ID NO: 6,
(c-2) a protein consisting of the amino acid sequence in which 1 to 20 amino acids are deleted, substituted or added in the amino acid sequence of SEQ ID NO: 6, and having trichodiene synthase activity, and
(c-3) a protein consisting of an amino acid sequence having homology of 90% or more with the amino acid sequence of SEQ ID NO: 6, and having trichodiene synthase activity.

Preferably, the first protein is cyclic diterpene synthase.

Preferably, the first protein is derived from prokaryote.

Preferably, the FKBP family protein is derived from prokaryote.

Preferably, the FKBP family protein is derived from archaea.

Preferably, the FKBP family protein has a molecular weight of 20,000 or less.

Preferably, the FKBP family protein is any one of the following (d-1) to (d-3):

(d-1) a protein consisting of the amino acid sequence of SEQ ID NO: 8,
(d-2) a protein consisting of the amino acid sequence in which 1 to 20 amino acids are deleted, substituted or added in the amino acid sequence of SEQ ID NO: 8, and having activity as the FKBP family protein, and
(d-3) a protein consisting of the amino acid sequence having homology of 90% or more with the amino acid sequence of SEQ ID NO: 8, and having activity as the FKBP family protein.

Preferably, the FKBP family protein belongs to a trigger factor.

Preferably, the FKBP family protein is linked to an N-terminal side of the first protein.

Preferably, the fusion protein further includes a secretion signal sequence.

One aspect of the present invention relates to a fusion protein encoded by the nucleic acid mentioned above.

One aspect of the present invention relates to a recombinant cell being a bacterium, including the above-mentioned nucleic acid, and expressing the fusion protein.

The recombinant cell of this aspect relates to a recombinant cell that is a bacterium, and expresses the fusion protein. According to the recombinant cell of this aspect, since the isoprene synthase activity or the cyclic terpene synthase activity is exhibited by the fusion protein, more stabilized isoprene synthase activity or cyclic terpene synthase activity is obtained.

One aspect of the present invention relates to a recombinant cell being a bacterium, including: a first nucleic acid encoding a first protein selected from the group consisting of isoprene synthase and cyclic terpene synthase; and a second nucleic acid encoding a FKBP family protein, wherein the recombinant cell expresses the isoprene synthase or the cyclic terpene synthase, and expresses the FKBP family protein.

The recombinant cell of this aspect is a bacterial recombinant cell that includes a first nucleic acid encoding isoprene synthase or cyclic terpene synthase, and a second nucleic acid encoding a FKBP family protein. According to the recombinant cell of this aspect, since the isoprene synthase or the cyclic terpene synthase is stabilized by a coexistent FKBP family protein, more stabilized isoprene synthase activity or cyclic terpene synthase activity is obtained.

Preferably, the first protein is isoprene synthase, and the recombinant cell expresses the isoprene synthase and the FKBP family protein.

The recombinant cell of this aspect includes a first nucleic acid encoding isoprene synthase, and a second nucleic acid encoding a FKBP family protein. According to the recombinant cell of this aspect, since the isoprene synthase is stabilized by a coexistent FKBP family protein, more stabilized isoprene synthase activity is obtained.

Preferably, the first protein is cyclic monoterpene synthase.

Preferably, the cyclic monoterpene synthase is phellandrene synthase.

Preferably, the first protein is cyclic sesquiterpene synthase.

Preferably, the first protein is cyclic diterpene synthase.

Preferably, the recombinant cell has a capability of assimilating a C1 compound.

Preferably, the recombinant cell has a capability of assimilating methanol or methane.

Preferably, the recombinant cell has a capability of assimilating carbon monoxide or carbon dioxide.

Preferably, the recombinant cell is an anaerobic bacterium.

Preferably, the recombinant cell is a Clostridium bacterium or a Moorella bacterium.

One aspect of the present invention relates to a method for producing isoprene or cyclic terpene, the method including: bringing gas that includes carbon dioxide and hydrogen into contact with the recombinant cell mentioned above; and allowing the recombinant cell to produce isoprene or cyclic terpene from carbon dioxide.

This aspect relates to a method for producing isoprene or cyclic terpene. The method includes bringing gas that includes carbon dioxide and hydrogen into contact with the recombinant cell above, and allowing the recombinant cell to produce isoprene or cyclic terpene from carbon dioxide. According to this aspect, since the isoprene synthase or cyclic terpene synthase expressed in the recombinant cell is stabilized by the FKBP family protein, isoprene or cyclic terpene is produced with high efficiency.

Preferably, the gas includes carbon monoxide, carbon dioxide and hydrogen, and the method allows the recombinant cell to produce isoprene or cyclic terpene from carbon monoxide and carbon dioxide.

This aspect also relates to a method for producing isoprene or cyclic terpene. The method includes bringing gas that includes carbon monoxide, carbon dioxide and hydrogen into contact with the above-mentioned recombinant cell, and allowing the recombinant cell to produce isoprene or cyclic terpene from carbon monoxide and carbon dioxide. Also, according to this aspect, since the isoprene synthase or cyclic terpene synthase expressed in the recombinant cell is stabilized by the FKBP family protein, isoprene or cyclic terpene is produced with high efficiency.

Preferably, isoprene or cyclic terpene released to the outside of the recombinant cell is collected.

Preferably, the isoprene or cyclic terpene is collected by a solid phase adsorption process.

Preferably, the isoprene or cyclic terpene is collected by a solvent absorption process.

### Effect of Invention

The present invention remarkably improves the productivity in production of isoprene or cyclic terpene by a recombinant cell.

### BRIEF DESCRIPTION OF DRAWINGS

FIG. 1 is an explanatory diagram showing an acetyl CoA pathway and a methanol pathway.

FIG. 2 is an explanatory diagram showing a carbon anabolic metabolic pathway via formaldehyde.

FIG. 3 is an explanatory diagram showing a structure of a pSKCL vector.

### BEST MODE FOR CARRYING OUT THE INVENTION

A nucleic acid (gene) of the present invention encodes a fusion protein (chimeric protein) in which isoprene synthase or cyclic terpene synthase is linked to a FKBP family protein. In other words, the nucleic acid of the present invention is a fusion gene (chimeric gene) in which a gene encoding isoprene synthase or cyclic terpene synthase and a gene encoding the FKBP family protein are linked to each other.

The FKBP family protein is a FK506 binding protein (FKBP), and has Peptidyl-prolyl cis-trans isomerase (hereinafter, referred to as “PPIase”) activity and molecular chaperone activity. The structures or classifications of the FKBP family proteins (FKBP-type PPIase) are described in, for example, WO2004/001041 and WO2005/063964.

Hereinafter, the FKBP family protein is also abbreviated as simply FKBP.

The PPIase activity is activity for catalyzing a cis-trans isomerization reaction of peptide bond at the N-terminal side of a proline residue in the protein. The molecular chaperone activity is activity of re-folding a denatured protein into an original normal type, or activity of inhibiting irreversible aggregation of a denatured protein.

The FKBP family protein has action of promoting folding speed of polypeptide based on the PPIase activity and action of suppressing insolubilization by interaction with hydrophobic peptide based on the molecular chaperone activity. The interaction with the hydrophobic peptide sequence with the FKBP family protein enables a nascent polypeptide to be free from degradation by protease.

An origin of the FKBP family protein used in the present invention is not particularly limited. For example, the FKBP family protein derived from prokaryote or archaea (archaebacteria) is used.

The FKBP family protein is generally classified into, depending on the molecular weight, a short type having a molecular weight of 20,000 or less, specifically, a molecular weight of about 16,000 to 18,000, and a long type having a molecular weight of about 26,000 to 33,000. In the present invention, the short type and the long type may be used. The short type is preferably used because the short type has higher molecular chaperone activity. That is to say, in the present invention, a FKBP family protein having a molecular weight of 20,000 or less is preferably used.

Furthermore, examples of FKBP family proteins other than those from archaea are classified mainly into a trigger factor type (Huang, Protein Sci. 9, 1254-, 2000), an FkpA type (Arie, Mol. Microbiol. 39, 199-, 2001), an FKBP52 type (Bose, Science 274, 1715-, 1996), and the like. Any types may be employed in the present invention. For example, the trigger factor type FKBP family protein is used. The FKBP family protein of the trigger factor type has been found in genomes of almost all the bacteria.

FKBP family proteins derived from archaea have been studied in detail (see, for example, WO2004/001041 and WO2005/063964). Examples of the short type FKBP family protein derived from archaea include FKBP family proteins derived from Methanococcus thermolithotrophicus, Thermococcus sp. KS-1, Methanococcus jannaschii, and the like (Maruyama, Front. Biosc. 5, 821-, 2000).

Examples of the long type FKBP family protein derived from archaea include FKBP family proteins derived from Pyrococcus horikoshii, Aeropyrum pemix, Sulfolobus solfataricus, Methanococcus jannaschii, Archaeoglobus fulgidus, Methanobacterium autotrophicum, Thermoplasma acidophilum, Halobacterium cutirubrum, and the like (Maruyama, Front. Biosci. 5, 821-, 2000).

As one example, SEQ ID NO: 7 shows a nucleotide sequence of a nucleic acid (DNA) encoding short type FKBP family protein (TcFKBP18) derived from Thermococcus sp. KS-1 and a corresponding amino acid sequence, and SEQ ID NO: 8 shows only the amino acid sequence.

The FKBP family protein used in the present invention may be not only a naturally occurring and isolated FKBP family protein but also a modified product thereof. For example, it may be proteins that are partial fragments of the existing FKBP family protein or may be amino acid substitution variants and having activity as FKBP family protein. The FKBP family protein activity includes PPIase activity and molecular chaperone activity.

As mentioned above, the PPIase activity is activity for catalyzing a cis-trans isomerization reaction of a peptide bond at the N-terminal side of the proline residue in the protein. Evaluation of the PPIase activity is carried out by, for example, a chymotrypsin coupling method (J. Bacteriol. 1998, 180(2): 388-394).

As mentioned above, the molecular chaperone activity means activity of re-folding a denatured protein into an original normal type, or activity of inhibiting irreversible aggregation of a denatured protein. For example, the molecular chaperone activity is evaluated as follows. That is, rhodanese, citrate synthetase, malate dehydrogenase, glucose-6-phosphate dehydrogenase, and the like, are used as model enzymes (Kawata, Bioscience and Industry 56, 593-, 1998), and these are denaturation-treated with a protein denaturing agent such as 6M guanidine hydrochloride. Then, molecular chaperone activity of a test substrate is evaluated based on a rate of regeneration of a denatured protein which is initiated upon dilution of a denaturing agent with a buffer containing the test substance, and a rate of inhibiting aggregation of a denatured protein. Examples of a method of assessing a rate of regeneration of a denatured protein include a method of Horowitz et al. (Horowitz, Methods Mol. Biol., 40, 361-, 1995) when rhodanese is used. Examples of a method of assessing inhibition of aggregation of a denatured protein include a method of Taguchi et al. (Taguchi, J. Biol. Chem., 269, 8529-, 1994).

For example, the FKBP family protein used in the present invention includes at least the following proteins (d-1) to (d-3):

(d-1) a protein consisting of the amino acid sequence of SEQ ID NO: 8,
(d-2) a protein consisting of the amino acid sequence in which 1 to 20 amino acids are deleted, substituted or added in the amino acid sequence of SEQ ID NO: 8, and having activity as the FKBP family protein, and
(d-3) a protein consisting of the amino acid sequence having homology of 90% or more with the amino acid sequence of SEQ ID NO: 8, and having activity as the FKBP family protein.

Note here that the homology of the amino acid sequence in (d-3) is more preferably 92% or more, further more preferably 95% or more, and particularly preferably 98% or more.

The homology of an amino acid sequence is calculated, for example, by using a multiple sequence alignment program such as CulustalW commercially available.

Next, isoprene synthase is described.

As mentioned above, the isoprene synthase (EC 4.2.3.27) has action of converting dimethylallyl diphosphate (DMAPP) as an isomer of isopentenyl diphosphate (IPP) into isoprene. Note here that structural conversion between IPP and DMAPP is catalyzed by isopentenyl diphosphate isomerase. The isopentenyl diphosphate isomerase is present in all organisms.

Hereinafter, the isoprene synthase is also abbreviated as simply IspS.

The isoprene synthase used in the present invention is not particularly limited. For example, isoprene synthase derived from eukaryote such as plant is used. General examples of the isoprene synthase derived from plants include the isoprene synthase derived from Populus nigra, Stizolobium deeringianum, and Pueraria lobata Ohwi. Other examples include isoprene synthase derived from genus Salix, genus Robinia, genus Wisteria, genus Triticum, genus Morus, and the like. All of them can be applied to all the present invention.

SEQ ID NO: 1 shows a nucleotide sequence of a nucleic acid (DNA) encoding the isoprene synthase derived from Populus nigra (GenBank Accession No.: AM410988.1) and a corresponding amino acid sequence. SEQ ID NO: 2 shows only the amino acid sequence.

Synthase derived from other than plants includes isoprene synthase derived from prokaryote. Examples thereof include isoprene synthase derived from Bacillus subtilis (Sivy T L. et al., Biochem. Biophys. Res. Commu. 2002, 294(1), 71-5).

Also the isoprene synthase used in the present invention may be not only a naturally occurring and isolated isoprene synthase but also a modified product thereof. For example, it may be proteins that are partial fragments of the existing isoprene synthase or may be amino acid substitution variants and having activity as isoprene synthase.

For example, the isoprene synthase used in the present invention includes at least the following (a-1) to (a-3):

(a-1) a protein consisting of the amino acid sequence of SEQ ID NO: 2,
(a-2) a protein consisting of the amino acid sequence in which 1 to 20 amino acids are deleted, substituted or added in the amino acid sequence of SEQ ID NO: 2, and having isoprene synthase activity, and
(a-3) a protein consisting of an amino acid sequence having homology of 90% or more with the amino acid sequence of SEQ ID NO: 2, and having isoprene synthase activity.

Note here that the homology of the amino acid sequence in (a-3) is more preferably 92% or more, further more preferably 95% or more, and particularly preferably 98% or more.

Next, cyclic terpene synthase is described.

As mentioned above, the cyclic terpene synthase is an enzyme for producing cyclic terpene using linear terpene as a substrate. Examples of the cyclic terpene synthase include cyclic monoterpene synthase, cyclic sesquiterpene synthase, cyclic diterpene synthase.

The cyclic monoterpene synthase has action of cyclizing geranyl diphosphate (GPP) to be converted into cyclic monoterpene.

Specific examples of the cyclic monoterpene synthase include phellandrene synthase. For example, β-phellandrene synthase that is one type of the phellandrene synthase has action of converting geranyl diphosphate (GPP) or neryl diphosphate (NPP) into β-phellandrene (Patent Document 5).

Specific examples of the β-phellandrene synthase and the nucleic acid encoding thereof includes β-phellandrene synthase derived from Solanum lycopersicum (GenBank Accession No.: FJ797957; Schilmiller, A. L., et al., Proc Natl Acad Sci USA., 2009, 106, 10865-70), β-phellandrene synthase derived from Lavandula angustifolia (GenBank Accession No.: HQ404305; Demissie, Z. A., et al., Planta, 2011, 233, 685-96), and the like.

SEQ ID NO: 3 shows a nucleotide sequence of a nucleic acid (DNA) encoding the β-phellandrene synthase derived from Lavandula angustifolia and a corresponding amino acid sequence. SEQ ID NO: 4 shows only the amino acid sequence. DNA having the nucleotide sequence set forth in SEQ ID NO: 3 is an example of a nucleic acid encoding the β-phellandrene synthase (cyclic monoterpene synthase).

The cyclic sesquiterpene synthase has action of cyclizing famesyl diphosphate (FPP) and converting thereof into cyclic sesquiterpene. Examples of the cyclic sesquiterpene synthase include trichodiene synthase.

SEQ ID NO: 5 shows a nucleotide sequence of a nucleic acid (DNA) encoding the trichodiene synthase derived from Fusarium poae and a corresponding amino acid sequence. SEQ ID NO: 6 shows only the amino acid sequence. DNA having the nucleotide sequence set forth in SEQ ID NO: 5 is an example of a nucleic acid encoding the trichodiene synthase (cyclic sesquiterpene synthase).

The cyclic diterpene synthase has action of cyclizing geranylgeranyl diphosphate (GGPP) to be converted into cyclic diterpene.

The cyclic terpene synthase used in the present invention may not only a naturally occurring and isolated cyclic terpene synthase but also a modified product thereof. For example, the cyclic terpene synthase may be a protein that is a partial fragment or an amino acid substituted variant of existing cyclic terpene synthase and that has cyclic terpene synthase activity.

For example, the cyclic monoterpene synthase used in the present invention includes at least the following (b-1) to (b-3):

(b-1) a protein consisting of the amino acid sequence of SEQ ID NO: 4,
(b-2) a protein consisting of the amino acid sequence in which 1 to 20 amino acids are deleted, substituted or added in the amino acid sequence of SEQ ID NO: 4, and having β-phellandrene synthase activity, and
(b-3) a protein consisting of an amino acid sequence having homology of 90% or more with the amino acid sequence of SEQ ID NO: 4, and having β-phellandrene synthase activity.

Note here that the homology of the amino acid sequence in (b-3) is more preferably 92% or more, further more preferably 95% or more, and particularly preferably 98% or more.

For example, the cyclic sesquiterpene synthase used in the present invention includes at least the following (c-1) to (c-3):

(c-1) a protein consisting of the amino acid sequence of SEQ ID NO: 6,
(c-2) a protein consisting of the amino acid sequence in which 1 to 20 amino acids are deleted, substituted or added in the amino acid sequence of SEQ ID NO: 6, and having trichodiene synthase activity, and
(c-3) a protein consisting of an amino acid sequence having homology of 90% or more with the amino acid sequence of SEQ ID NO: 6, and having trichodiene synthase activity.

Note here that the homology of the amino acid sequence in (c-3) is more preferably 92% or more, further more preferably 95% or more, and particularly preferably 98% or more.

In a fusion protein encoded by the nucleic acid of the present invention, the link direction of the first protein (isoprene synthase or cyclic terpene synthase) and the FKBP family protein is not particularly limited. That is to say, the FKBP family protein may be linked to the N-terminal side of the first protein, or the FKBP family protein may be linked to the C-terminal side of the first protein. Furthermore, the first protein and the FKBP family protein may be linked to each other directly, or may be linked to each other via a peptide linker or the like. As the peptide linker, for example, a peptide linker that includes a structurally flexible amino acid sequence consisting of about 10 to 50 amino acids is used. For example, a peptide linker consisting of repeating structure of amino acid sequence of four glycines and one serine is used.

The fusion protein may further include a secretion signal. When the secretion signal is provided, the fusion protein can be secreted when it is expressed in a host cell.

The present invention encompasses a fusion protein encoded by the above-mentioned nucleic acid, that is, a fusion protein in which the first protein (isoprene synthase or cyclic terpene synthase) and the FKBP family protein are linked to each other

The present invention encompasses a recombinant cell that is a bacterium, includes a nucleic acid encoding the above-mentioned fusion protein, and expresses the fusion protein.

Furthermore, the present invention encompasses a recombinant cell that is a bacterium, includes a first nucleic acid encoding a first protein (isoprene synthase or cyclic terpene synthase) and a second nucleic acid encoding a FKBP family protein, and expresses the isoprene synthase or cyclic terpene synthase and the FKBP family protein.

In addition, the present invention encompasses a recombinant cell that is a bacterium, includes a first nucleic acid encoding isoprene synthase and a second nucleic acid encoding a FKBP family protein, and expresses the isoprene synthase and the FKBP family protein.

In any recombinant cells, isoprene synthase or cyclic terpene synthase is stabilized by the presence of adjacent or coexistent FKBP family protein.

A recombinant cell of the present invention is obtained, for example, by introducing the nucleic acid into a host cell such as a bacterium.

In general, prokaryotes such as bacteria have a capability of synthesizing isopentenyl diphosphate (IPP) by a non-mevalonate pathway (MEP pathway). Furthermore, as mentioned above, the isopentenyl diphosphate isomerase is present in all organisms. Therefore, the recombinant cell of the present invention converts DMAPP converted from IPP into isoprene. That is to say, the recombinant cell of the present invention produces isoprene.

Furthermore, the recombinant cell of the present invention converts geranyl diphosphate (GPP), farnesyl diphosphate (FPP), geranylgeranyl diphosphate (GGPP), and the like, having IPP as a precursor, into cyclic terpene. That is to say, the recombinant cell of the present invention produces cyclic terpene.

The host cell is not particularly limited as long as it is bacteria. For example, bacteria used as a host for allowing a foreign gene to express, for example, Escherichia coli and Bacillus subtilis are applied also in the present invention.

As one embodiment, bacteria having a capability of assimilating a C1 compound are used as a host cell. For example, bacteria having a capability of assimilating carbon monoxide, carbon dioxide, methane, methanol, methyl amine, formic acid, formamide, and the like, are used as a host cell. Thus, a recombinant cell having a capability of assimilating a C1 compound such as carbon monoxide, carbon dioxide, methane, methanol, methyl amine, formic acid, and formamide is obtained.

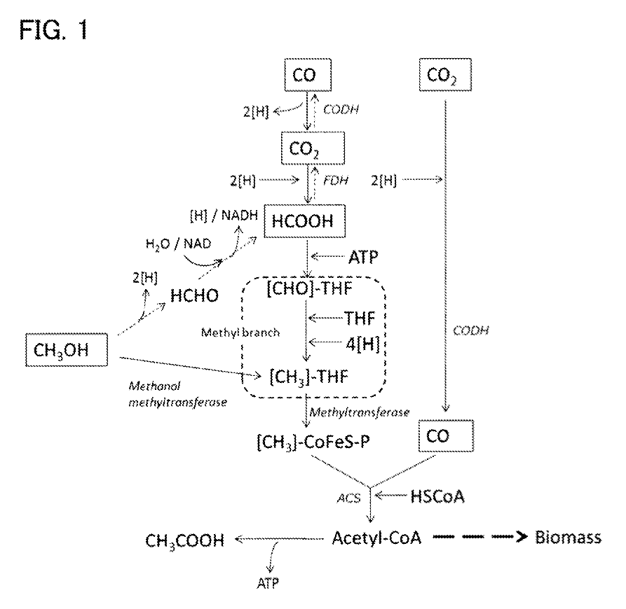

In one embodiment, bacteria having a capability of assimilating carbon monoxide or carbon dioxide are used as a host cell. This makes it possible to obtain a recombinant cell having a capability of assimilating carbon monoxide or carbon dioxide. Specific examples of the host cell include an anaerobic prokaryotic cell. In particular, examples of the host cell include an anaerobic prokaryotic cell having a function of synthesizing acetyl CoA from methyl tetrahydrofolate, carbon monoxide, and CoA. Furthermore, an anaerobic prokaryotic cell further including carbon monoxide dehydrogenase (EC 1.2.99.2) is preferable. In detail, the preferable host cell is an anaerobic prokaryotic cell, which grows mainly by carbon monoxide metabolism, that is, a function of generating carbon dioxide and proton from carbon monoxide and water by the action of the carbon monoxide dehydrogenase. Examples of such an anaerobic prokaryotic cell includes an anaerobic prokaryotic cell having an acetyl CoA pathway (Wood-Ljungdahl pathway) and a methanol pathway shown in FIG. 1.

Use of such an anaerobic prokaryotic cell as a host cell makes it possible to allow a recombinant cell to produce isoprene or cyclic terpene using gas such as carbon monoxide or carbon dioxide as a carbon source.

Examples of the anaerobic prokaryotic cell include a Clostridium bacterium or a Moorella bacterium, for example, Clostridium ljungdahlii, Clostridium autoethanogenumn, Clostridium carboxidivorans, Clostridium ragsdalei (Kopke M. et al., Appl. Environ. Microbiol. 2011, 77(15), 5467-5475), Moorella thermoacetica (same as Clostridium thermoaceticum) (Pierce E G. Et al., Environ. Microbiol. 2008, 10, 2550-2573). In particular. Clostridium bacteria are preferable as the host cell in the recombinant cell of the present invention because their host-vector systems and culture methods have been established. Note here that the five species of Clostridium bacteria or Moorella bacteria mentioned above are known as representative examples of syngas assimilating microorganisms.

Besides, prokaryotic cells such as Carboxydocella sporoducens sp. Nov., Rhodopseudomonas gelatinosa, Eubacterium limosum, Butyribacterium methylotrophicum, and the like, are used as the host cell.

A basic configuration of a technique for allowing a recombinant cell to produce isoprene from syngas is described in the above mentioned Patent Document 1.

As one embodiment, a methanol assimilating bacterium as one type of methylotroph is used as the host cell. This makes it possible to obtain a recombinant cell having methanol assimilating ability.

A methylotroph is a general name for a C1 compound assimilating microorganism that uses a carbon compound that does not have a C—C bond in the molecule, for example, methane, methanol, methylamine, dimethylamine, trimethylamine, or the like, as a sole carbon source or an energy source. All of the microorganisms called methanotroph, methane-oxidizing bacteria, methanol assimilating bacteria, methanol assimilating yeast, methanol assimilating microorganisms belong to methylotrophs. Methanotroph converts methane into methanol by the action of methane monooxygenase, and dissimilates methanol by the same metabolism as in methyltroph.

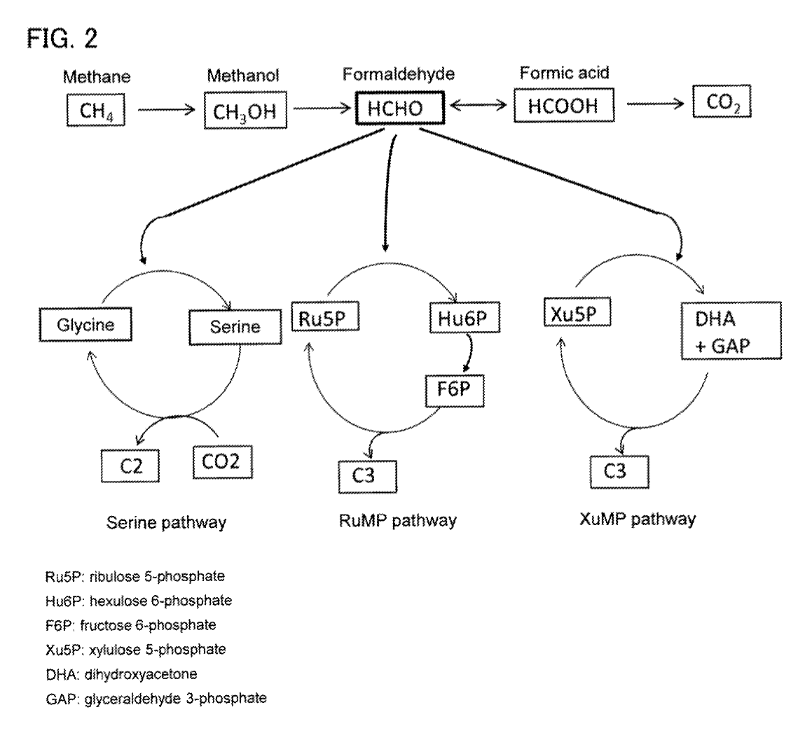

Central metabolism of methylotroph is a reaction of converting methanol into formaldehyde and then converting formaldehyde into an organic compound having a C—C bond. As a carbon assimilation metabolism pathway via formaldehyde, a serine pathway, a ribulose monophosphate pathway (RuMP pathway), and a xylulose monophosphate pathway (XuMP pathway) shown in FIG. 2 are known. Methanol assimilating bacteria used in the present invention have a serine pathway or a RuMP pathway.

Methylotroph has a function of converting methanol and/or formic acid into formaldehyde and a capability of fixing formaldehyde.

Examples of the methanol assimilating bacteria include methylotroph belonging to the genus Methylacidphilum, the genus Methylosinus, the genus Methylocystis, the genus Methylobacterium, the genus Methylocella, the genus Methylococcus, the genus Methylomonas, the genus Methylobacter, the genus Methylobacillus, the genus Methylophilus, the genus Methylotenera, the genus Methylovorus, the genus Methylomicrobium, the genus Methylophaga, the genus Methylophilaceae, the genus Methyloversatllis, the genus Mycobacterium, the genus Arthrobacter, the genus Bacillus, the genus Beggiatoa, the genus Burkholderia, the genus Granulibacter, the genus Hyphomicrobium, the genus Pseudomonas, the genus Achromobactor, the genus Paracoccus, the genus Crenothrix, the genus Clonothrix, the genus Rhodobacter, the genus Rhodocyclaceae, the genus Silicibacter, the genus Thiomicrospira, and the genus Verrucomicrobia.

Bacteria other than methylotroph can be treated as methanol assimilating bacteria by imparting “a function of converting methanol and/or formic acid into formaldehyde” and “formaldehyde fixing ability”, which are characteristics of the methylotroph.

A basic configuration of a technique for allowing a recombinant cell as methylotroph to produce isoprene from methanol or methane is described in the above mentioned Patent Document 2.

The method of introducing a gene into the host cell (bacterium) may be selected appropriately depending on the type of the host cell and the like. For example, a method for introducing a target gene by a self-replicating plasmid (vector) or a genome introduction method is used.

For example, an self-replicating plasmid (vector) capable of being introduced into the host cell and capable of expressing the incorporated nucleic acid is used. A vector that autonomously replicates or is incorporated in chromosome in the host cell, and contains a promoter at the position allowing transcription of the inserted nucleic acid (DNA) may be used. For example, it is preferred to construct in the host cell a series of structures including a promoter, a ribosome binding sequence, the above nucleic acid (DNA) and a transcription termination sequence by using the vector.

For introducing a plurality of types of nucleic acids into the host cell by using a vector, the nucleic acids may be incorporated in one vector, or incorporated in individual vectors. When a plurality of types of nucleic acids are incorporated in one vector, these nucleic acids may be expressed under a common promoter for these nucleic acids, or expressed under individual promoters. Examples of introducing a plurality of types of nucleic acids include an embodiment of introducing a nucleic acid encoding the FKBP family protein and a nucleic acid encoding isoprene synthase or cyclic terpene synthase (co-expression).

Examples of the self-replicating vectors to be used when the host is Escherichia coli include commercially available pET (available from Novagen), pBAD (available from Life technologies), pGEM (available from Promega), and the like. Examples of vectors when the host is a methanol assimilating bacterium include pAYC32 (Chistoserdov A Y., et al., Plasmid 1986, 16, 161-167), pRP301 (Lane M., et al., Arch. Microbiol. 1986, 144(1), 29-34), pBBR1, pBHR1 (Antoine R. et al., Molecular Microbiology 1992, 6, 1785-1799), pCM80 (Marx C J. et al., Microbiology 2001, 147, 2065-2075), and the like. Examples of vectors when the host is Clostridium bacterium include pJIR (Brandshaw M., et al., Plasmid 40 (3), 233-237), pIMP1 (Mermelstein L D et al., Bio/technology 1992, 10, 190-195), pMTL (Ng, Y K. Et al., PLoS One 2013, 8 (2), e56051), pMVTcatMCS47 (Berzin, V. et al., Appl. Biochem. Biotechnol. 2012, 167, 338-347), and the like. These self-replicating vectors are introduced into a host cell by electroporation, a joining method, a chemical treatment method, and the like.

On the other hand, the genome introduction method is carried out using techniques such as homologous recombination, a transposon method (Martinerz-Garcia E. et al., BMC Microbiol. 11:38), an integrase method (Miyazaki, R. et al., Appl. Environ. Microbiol. 2014, 79 (14), 4440-4447), a Cre/loxP method (Bertram, R. et al., J. Mol. Microbiol. Biotechnol. 2009, 17 (3), 136-145), a Flp/FRT method (Al-Hinai, M A. et al., Appl. Environ. Microbiol. 2012, 78 (22), 8112-8121), and the like.

From the viewpoint of improving the production ability of isoprene or cyclic terpene, other nucleic acids may be further introduced into the recombinant cell. For example, a nucleic acid encoding enzyme (enzyme group) acting on a mevalonate pathway (MVA pathway) or non-mevalonate pathway (MEP pathway) as a synthesis pathway of isopentenyl diphosphate (IPP) is introduced. Thus, synthesis ability of IPP as a supply source of DMAPP is enhanced, resulting in enhancing synthesis ability of isoprene or cyclic terpene.

The method for culturing the recombinant cell of the present invention is not particularly limited. The recombinant cell may be cultured in a medium that allows the recombinant cell to proliferate. For example, when the host is heterotrophic microorganism such as Escherichia coli and Bacillus subtilis, carbon sources such as glucose and saccharose are used. When the host is methanol assimilating bacteria, a medium containing 0.1 to 5.0/0 (v/v) methanol is preferably used. When the host is syngas-assimilating bacteria, the followings can be used as carbon sources and energy sources.

### CO

### CO/H₂

CO/CO₂/H₂

### CO/HCOOH

### CO₂/HCOOH

### CO/CH₃OH

CO₂/CH₃OH

### CO/H₂/HCOOH

CO₂/H₂/HCOOH
CO/H₂/CH₃OH
CO₂/H₂/CH₃OH
CO/CO₂/H₂/HCOOH
CO/CO₂/H₂/CH₃OH
CH₃OH/H₂

### HCOOH/H₂

Furthermore, even when the host is methanol-assimilating bacteria or syngas-assimilating bacteria, a sugar carbon source such as glucose and saccharose may be added in culture if necessary. Furthermore, the culture may be carried out by any methods such as a batch culture method, a fed-batch culture method, or a continuous culture method. In the fed-batch culture or the continuous culture, the culture solution is subjected to semipermeable membrane circulation, so that culture can be carried out with the cell density enhanced.

The present invention encompasses a method for producing isoprene or cyclic terpene by bringing gas that contains carbon dioxide and hydrogen into contact with the above-mentioned recombinant cell, and allowing the recombinant cell to produce isoprene or cyclic terpene from carbon dioxide. Furthermore, the present invention encompasses a method for producing isoprene or cyclic terpene by bringing gas that contains carbon monoxide, carbon dioxide and hydrogen into contact with the above-mentioned recombinant cell, and allowing the recombinant cell to produce isoprene or cyclic terpene from the carbon monoxide and carbon dioxide.

For example, a recombinant cell using an anaerobic prokaryotic cell having a function of synthesizing acetyl CoA from methyl tetrahydrofolate, carbon monoxide, and CoA mentioned above as a host cell is brought into contact with the above-mentioned gas, and isoprene or cyclic terpene is produced from the above-mentioned gas.

As one example, the recombinant cell is cultured by using the above-mentioned gas as a carbon source, and isoprene or cyclic terpene is isolated from the cultured product.

Besides, isoprene or cyclic terpene is produced by bringing the above-mentioned gas into contact with the recombinant cell regardless of whether or not cell proliferation occurs. For example, the above-mentioned gas is continuously fed to the fixed recombinant cell, so that isoprene or cyclic terpene can be produced continuously.

The isoprene or cyclic terpene produced by a recombinant cell is collected from, for example, an extracellular vapor phase. Examples of the collecting method may include a solid phase adsorption process and a solvent absorption process.

Note here that when the recombinant cell of the present invention is cultured exclusively for cell proliferation, or obtaining a fusion protein itself, rather than for production of isoprene or cyclic terpene isoprene, it is not necessary to use the above-mentioned gas as a carbon source. For example, the recombinant cell may be cultured using other carbon sources such as saccharides or glycerin as mentioned above.

In the following, the present invention will be described more specifically by way of examples. However, the present invention is not limited to these examples.

### EXAMPLE

### Example 1

Expression of FKBP-IspS Fusion Protein in Escherichia coli BL21 Strain (Lon⁻, OmpT⁻) and Isoprene Production

An artificial synthetic gene (DNA) of SEQ ID NO: 9 was prepared. The artificial synthetic gene includes a gene encoding a fusion protein consisting of FKBP derived from Thermococcus and isoprene synthase (IspS) derived from Populus nigra, and a gene encoding isopentenyl diphosphate isomerase (IDI) derived from actinomycete. Furthermore, the artificial synthetic gene is designed to have a tag sequence composed of six histidine residues at the C-terminal of the FKBP-IspS fusion protein. The artificial synthetic gene was introduced into NdeI and BamHI cleavage sites of a pET-3a vector to construct an expression vector pTFKIS. pTFKIS was introduced into an Escherichia coli BL21 strain (Lon⁻, OmpT⁻) to obtain a recombinant.

The recombinant was cultured at 30° C. in a 2×YT medium containing 100 μg/mL ampicillin. After 16 hours from the start of culture, 5 mL of culture solution was transferred to TORAST HS 20 ml vial (manufactured by SHIMADZU CORPORATION) scaled by a septum cap, IPTG was added thereto at 0.1 mM, and further continued to be cultured at 30° C. for 20 hours. After the culture, headspace sampling measurement using GCMS QP2010 ultra (SHIMADZU CORPORATION) was carried out. As a GC column, ZB-624 (manufactured by phenomenex: membrane thickness: 1.40 μm/length: 30.0 m/inner diameter: 0.25 mm) was used. An amount of isoprene production per dry cell weight (g) was calculated. Culture was carried out three times in total, and the mean value thereof was employed (N=3).

On the other hand, after the completion of culture, the cells were collected, and subjected to ultrasonic treatment to obtain a cell disruption solution. The cell disruption solution was centrifuged and separated into a supernatant (a soluble fraction) and a precipitated fraction. Both these fractions were subjected to electrophoresis, and expression of FKBP-IspS fusion protein was assessed by Western blotting using anti-6 His-tag antibody (manufactured by GE Healthcare). The evaluation of the expression level was carried out by evaluating the color intensity of antibody staining in six stages of 0 to 5.

As a control, the same experiment was carried out with respect to Escherichia coli BL21 strain (Lon⁻, OmpT⁻) in which only a pET3a vector had been introduced.

### Comparative Example 1

The same experiment was carried out as in Example 1 except that IspS was singly used instead of using a FKBP-IspS fusion protein.

An artificial synthetic gene (DNA) of SEQ ID NO: 10 was prepared. The artificial synthetic gene includes a gene encoding IspS derived from Populus nigra, and a gene encoding isopentenyl diphosphate isomerase (IDI) derived from actinomycete, but it does not include an FKBP gene. Furthermore, the artificial synthetic gene is designed to have a tag sequence having six histidine residues at the C-terminal of IspS. The artificial synthetic gene was introduced into NdeI and BamHI cleavage sites of pET-3a vector to construct an expression vector pTIS. pTIS was introduced into an Escherichia coli BL21 strain (Lon⁻, OmpT⁻) to obtain a recombinant.

The recombinant was cultured in the same manner as in Example 1. Furthermore, headspace sampling measurement was carried out in the same manner as in Example 1. Furthermore, the expression of IspS was assessed by Western blotting.

Results are shown in Table 1. That is to say, an amount of isoprene production by an Escherichia coli recombinant (Example 1) into which a gene encoding a fusion protein of FKBP and IspS had been introduced was 54.5 mg per gram of dry cell, which was significantly higher than an amount of isoprene production (7.4 mg per gram of dry cell) by an Escherichia coli recombinant into which an IspS gene as a non-fused gene had been introduced (Comparative Example 1).

Furthermore, in the expression of IspS (color intensity in Western blotting), in the recombinant of Example 1, a sufficient amount of IspS was detected in the soluble fraction, and only a slight amount of IspS was detected in the precipitated fraction. This showed that when IspS was fused with FKBP, the expression level of the active IspS holding a normal three-dimensional structure in the soluble fraction was increased, thus increasing the isoprene production. On the other hand, in the recombinant of Comparative Example 1, IspS was detected mainly in the precipitated fraction, only a small amount of IspS was detected in the soluble fraction.

As mentioned above, use of Escherichia coli recombinant into which a gene encoding a fusion protein of FKBP and IspS had been introduced was able to significantly improve the amount of isoprene production.

TABLE 1

| | | Isoprene | | |
|---|---|---|---|---|
| | | production | Color intensity in | |
| Vector | Introduced gene | (mg/DCW*) | Western blotting | |
| pTFKIS | FKBP-IspS-IDI | 54.5 | Soluble | 5 |
| (Example 1) | | | fraction | |
| | | | Precipitated | 1 |
| | | | fraction | |
| pTIS | IspS-IDI | 7.4 | Soluble | 2 |
| (Comparative | | | fraction | |
| Example 1) | | | Precipitated | 4 |
| | | | fraction | |
| pET3a | — | Not detected | Soluble | 0 |
| | | | fraction | |
| | | | Precipitated | 0 |
| | | | fraction | |

*DCW: Dry cell weight (g)

### Example 2

### Expression of FKBP-IspS Fusion Protein in Syngas-Assimilating Bacterium Clostridium Ljungdahlii and Isoprene Production

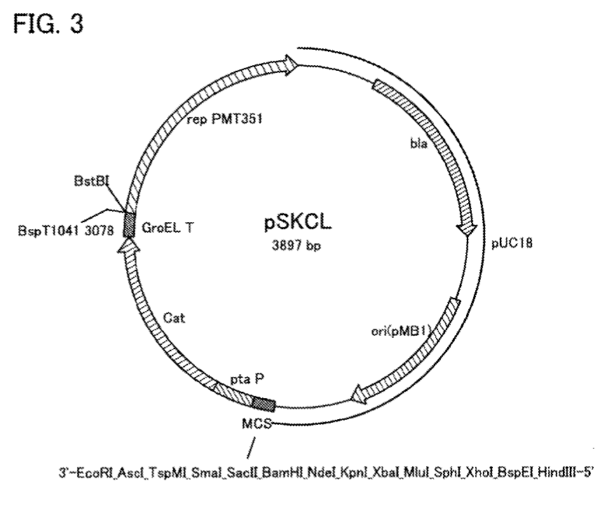

An artificial synthetic gene (DNA) of SEQ ID NO: 11 was prepared. As in Example 1, the artificial synthetic gene includes a gene encoding a fusion protein consisting of FKBP derived from Thermococcus and IspS derived from Populous nigra, and a gene encoding IDI derived from actinomycete, and designed to induce expression of these genes by a pta (Phosphotransacetylase) promoter derived from C. ljungdahlii. Furthermore, the artificial synthetic gene is designed to have a tag sequence composed of six histidine residues at the C-terminal of the FKBP-IspS fusion protein. The artificial synthetic gene was introduced into TspMI and BspEI cleavage sites of a pSKCL vector (FIG. 3, SEQ ID NO: 13) to construct an expression vector pSCLFKIS. pSCLFKIS was introduced into a C. ljungdahlii (DSM13528) strain to obtain a recombinant.

The recombinant was cultured at 37° C. in 5 mL of PETC 1754 medium (ATCC: American Type Culture Collection) containing 30 μg/mL chloramphenicol in TORAST HS 20 ml vial (manufactured by SHIMADZU CORPORATION) sealed by a septum cap. At the time when the OD600 value of the culture solution exceeded 1.0, an isoprene amount in a vapor phase was measured in the same manner as in Example 1. Culture was carried out three times in total, and the mean value thereof was employed (N=3).

Furthermore, after culture, the cells were collected, and subjected to French press to obtain a cell disruption solution. The cell disruption solution was centrifuged and separated into a supernatant (a soluble fraction) and a precipitated fraction. Both the fractions were subjected to Western blotting in the same manner as in Example 1 to assess the expression of IspS.

As a control, the same experiment was carried out also with respect to C. ljungdahlii into which only a pSKCL vector had been introduced.

### Comparative Example 2

The same experiment was carried out as in Example 2 except that IspS was singly used instead of using FKBP-IspS fusion protein.

An artificial synthetic gene (DNA) of SEQ ID NO: 12 was prepared. The artificial synthetic gene includes a gene encoding IspS derived from Populus nigra, and a gene encoding isopentenyl diphosphate isomerase (IDI) derived from actinomycete, but it does not include an FKBP gene. Furthermore, the artificial synthetic gene is designed to induce expression of these genes by a pta promoter derived from C. ljungdahlii. Furthermore, the artificial synthetic gene is designed to have a tag sequence having six histidine residues at the C-terminal of FKBP-IspS fusion protein. The artificial synthetic gene was introduced into TspMI and BspEI cleavage sites of pSKCL vector (FIG. 3) to construct an expression vector pSCLIS. pSCLIS was introduced into a C. ljungdahlii (DSM13528) strain to obtain a recombinant.

The recombinant was cultured in the same manner as in Example 2. Furthermore, headspace sampling measurement was carried out in the same manner as in Example 2. Furthermore, the expression of IspS was assessed by Western blotting.

Results are shown in Table 2. That is to say, an amount of isoprene production by a C. ljungdahlii recombinant (Example 2) into which a gene encoding a fusion protein of FKBP and IspS had been introduced was 2963 μg per gram of dry cell, which was significantly higher than an amount of isoprene production (0.50 μg per gram of dry cell) by a C. ljungdahlii recombinant into which an IspS gene as a non-fused gene had been introduced (Comparative Example 2).

Furthermore, in the expression of IspS (color intensity in Western blotting), in the recombinant of Example 2, a sufficient amount of IspS was detected in the soluble fraction, and only a slight amount of IspS was detected in the precipitated fraction. On the contrary, in the recombinant of Comparative Example 2, IspS was hardly detected, and it was slightly observed in the precipitated fraction. This was thought to be because in C. ljungdahlii, non-fused IspS was not able to form a normal three-dimensional structure, and almost all was degraded by protease.

As mentioned above, use of C. ljungdahlii recombinant into which a gene encoding a fusion protein FKBP and IspS had been introduced made it possible to remarkably improve the amount of isoprene production.

TABLE 2

| | | Isoprene | | |
|---|---|---|---|---|
| | | production | Color intensity in | |
| Vector | Introcuced gene | (mg/DCW*) | Western blotting | |
| pSCLFKIS | FKBP-IspS-IDI | 2963 | Soluble | 4 |
| (Example 2) | | | fraction | |
| | | | Precipitated | 1 |
| | | | fraction | |
| pSCLIS | IspS-IDI | 0.50 | Soluble | 0 |
| (Comparative | | | fraction | |
| Example 2) | | | Precipitated | 1 |
| | | | fraction | |
| pSKCL | — | 0.36 | Soluble | 0 |
| | | | fraction | |
| | | | Precipitated | 0 |
| | | | fraction | |

*DCW: Dry cell weight (g)

### Example 3

Expression of FKBP-β-Phellandrene Fusion Protein in Methylotroph Methylobacterium extorquens and Production of β-Phellandrene

In this Example, recombinant Methylobacterium extorquens for producing β-phellandrene as one type of cyclic monoterpene was prepared. Note here that M. extorquens holds an enzyme for synthesizing geranyl diphosphate (GPP), which is a substrate of β-phellandrene synthase, from isopentenyl diphosphate (IPP). Therefore, it was thought that when a β-phellandrene synthase gene was introduced into this strain, and the gene was allowed to express, thus enabling β-phellandrene to be synthesized from methanol.

An artificial synthetic gene (DNA) of SEQ ID NO: 14 was prepared. The artificial synthetic gene includes a gene encoding a fusion protein consisting of FKBP derived from Thermococcus and β-phellandrene synthase (bPHS) derived from Lavandula angustifolia, and designed to induce expression of the gene by the pta promoter used in Example 2. The artificial synthetic gene was introduced into a BamHI/KpnI site of pCM80 (Marx C J. et al., Microbiology 2001, 147, 2065-2075) set forth in SEQ ID NO: 15 as a broad host range vector to prepare pC80FkPHS. The expression vector pCM80FkPHS was introduced into M. extorquens by electroporation to obtain a ME-FkPHS strain.

The ME-FkPHS strain was cultured aerobically at 30° C. in 20 mL, of synthetic medium containing methanol as a sole carbon source (1 L of the synthetic medium contains 18 g of H₃PO₄, 14.28 g of K₂SO₄, 3.9 g of KOH, 0.9 g of CaSO₄.2H₂O, 11.7 g of MgSO₄.7H₂O, 8.4 mg of CuSO₄.5H₂O, 1.1 mg of KI, 4.2 mg of MnSO₄H₂O, 0.3 mg of NaMoO₄.2H₂O, 0.03 mg of H₃BO₃, 0.7 mg of CoCl₂.6H₂O, 28 mg of ZnSO₄.7H₂O, 91 mg of FeSO₄.7H₂O, 0.28 mg of biotin, 5 mL of methanol, and 10 mg of tetracycline). At the time when the OD600 value of the culture solution was 1.0 to 1.2, cells were collected. A part of the collected cells was cultured at 30° C. in 5 mL of the above-mentioned synthetic medium containing 20 μg/mL tetracycline in TORAST HS 20 ml vial (manufactured by SHIMADZU CORPORATION) sealed by a septum cap. At the time when the OD600 value of the culture solution exceeded 1.0, a β-phellandrene amount in a vapor phase was measured in the same manner as in Example 1. Culture was carried out three times in total, and the mean value thereof was employed (N=3).

As a control, the same experiment was carried out also in M. extorquens into which only a pCM80 vector had been introduced.

As a result, M. extorquens into which pCM80 had been introduced did not produce β-phellandrene at all, but the ME-FkPHS strain produced 7.3 mg per gram of dry cell of β-phellandrene.

### Comparative Example 3

The same experiment was carried out as in Example 3 except that bPHS was singly used instead of a FKBP-bPHS fusion protein.

An artificial synthetic gene of SEQ ID NO: 16 was prepared. The artificial synthetic gene includes a gene encoding only β-phellandrene synthase (bPHS) derived from Lavandula angustifolia. The artificial synthetic gene is designed to induce expression of the gene by the pta promoter used in Example 3. The artificial synthetic gene was introduced into a BamHI/KpnI site of pCM80 used in Example 3 to prepare pC80PHS. The expression vector pCM80PHS was introduced into M. extorquens by electroporation to obtain an ME-PHS strain. Culture was carried out in the same manner as in Example 3.

As a result, an amount of β-phellandrene production by ME-PHS strain was 0.4 mg per gram of dry cell. From the above-mentioned results, the amount of β-phellandrene production was able to be remarkably increased by fusing FKBP and bPHS also in methylotroph.

1. A nucleic acid encoding a fusion protein, the fusion protein comprising:
a first protein selected from the group consisting of isoprene synthase and cyclic terpene synthase; and
a FKBP family protein linked to the first protein.

2. The nucleic acid according to claim 1, wherein the first protein is isoprene synthase.

3. The nucleic acid according to claim 2, wherein the isoprene synthase is any one of the following (a-1) to (a-3):
(a-1) a protein consisting of the amino acid sequence of SEQ ID NO: 2,
(a-2) a protein consisting of the amino acid sequence in which 1 to 20 amino acids are deleted, substituted or added in the amino acid sequence of SEQ ID NO: 2, and having isoprene synthase activity, and
(a-3) a protein consisting of an amino acid sequence having homology of 90% or more with the amino acid sequence of SEQ ID NO: 2, and having isoprene synthase activity.

4. The nucleic acid according to claim 1, wherein the first protein is cyclic monoterpene synthase.

5. The nucleic acid according to claim 4, wherein the cyclic monoterpene synthase is phellandrene synthase.

6. The nucleic acid according to claim 4, wherein the cyclic monoterpene synthase is any one of the following (b-1) to (b-3):
(b-1) a protein consisting of the amino acid sequence of SEQ ID NO: 4,
(b-2) a protein consisting of the amino acid sequence in which 1 to 20 amino acids are deleted, substituted or added in the amino acid sequence of SEQ ID NO: 4, and having β-phellandrene synthase activity, and
(b-3) a protein consisting of an amino acid sequence having homology of 90% or more with the amino acid sequence of SEQ ID NO: 4, and having β-phellandrene synthase activity.

7. The nucleic acid according to claim 1, wherein the first protein is cyclic sesquiterpene synthase.

8. The nucleic acid according to claim 7, wherein the cyclic sesquiterpene synthase is any one of the following (c-1) to (c-3):
(c-1) a protein consisting of the amino acid sequence of SEQ ID NO: 6,
(c-2) a protein consisting of the amino acid sequence in which 1 to 20 amino acids are deleted, substituted or added in the amino acid sequence of SEQ ID NO: 6, and having trichodiene synthase activity, and
(c-3) a protein consisting of an amino acid sequence having homology of 90% or more with the amino acid sequence of SEQ ID NO: 6, and having trichodiene synthase activity.

9. The nucleic acid according to claim 1, wherein the first protein is cyclic diterpene synthase.

10. The nucleic acid according to claim 1, wherein the first protein is derived from prokaryote.

11. The nucleic acid according to claim 1, wherein the FKBP family protein is derived from prokaryote.

12. The nucleic acid according to claim 1, wherein the FKBP family protein is derived from archaea.

13. The nucleic acid according to claim 1, wherein the FKBP family protein has a molecular weight of 20,000 or less.

14. The nucleic acid according to claim 1, wherein the FKBP family protein is any one of the following (d-1) to (d-3):
(d-1) a protein consisting of the amino acid sequence of SEQ ID NO: 8,
(d-2) a protein consisting of the amino acid sequence in which 1 to 20 amino acids are deleted, substituted or added in the amino acid sequence of SEQ ID NO: 8, and having activity as the FKBP family protein, and
(d-3) a protein consisting of the amino acid sequence having homology of 90% or more with the amino acid sequence of SEQ ID NO: 8, and having activity as the FKBP family protein.

15-18. (canceled)

19. A recombinant cell being a bacterium, comprising the nucleic acid according to claim 1, and expressing the fusion protein.

20. A recombinant cell being a bacterium, comprising:
a first nucleic acid encoding a first protein selected from the group consisting of isoprene synthase and cyclic terpene synthase; and
a second nucleic acid encoding a FKBP family protein,
wherein the recombinant cell expresses the isoprene synthase or the cyclic terpene synthase, and expresses the FKBP family protein.

21-25. (canceled)

26. The recombinant cell according to claim 19, having a capability of assimilating a C1 compound.

27-30. (canceled)

31. A method for producing isoprene or cyclic terpene, the method comprising:
bringing gas that includes carbon dioxide and hydrogen into contact with the recombinant cell according to claim 26; and
allowing the recombinant cell to produce isoprene or cyclic terpene from carbon dioxide.

32-35. (canceled)

36. The recombinant cell according to claim 20, having a capability of assimilating a C1 compound.

37. A method for producing isoprene or cyclic terpene, the method comprising:
bringing gas that includes carbon dioxide and hydrogen into contact with the recombinant cell according to claim 36; and
allowing the recombinant cell to produce isoprene or cyclic terpene from carbon dioxide.
